(19) United States
(12) Patent Application Publication
(10) Pub. No.: US 2024/0059695 A1
(43) Pub. Date: Feb. 22, 2024

(54) SUBSTITUTED IMIDAZO[1,5-b]PYRIDAZINE COMPOUNDS AS KINASE INHIBITORS AND USE THEREOF

(71) Applicant: IMPACT THERAPEUTICS (SHANGHAI), INC, Shanghai (CN)

(72) Inventors: Sui Xiong CAI, Shanghai (CN); Ye Edward TIAN, Shanghai (CN); Xiaozhu WANG, Nanjing, Jiangsu (CN)

(21) Appl. No.: 18/259,112

(22) PCT Filed: Dec. 24, 2021
(86) PCT No.: PCT/CN2021/141153

§ 371 Date: Jun. 23, 2023

(30) Foreign Application Priority Data
Dec. 25, 2020 (CN) 202011572507.6

(51) Int. Cl. C07D 487/04; A61K 45/06; A61P 35/00

(52) U.S. Cl. CPC C07D 487/04; A61K 45/06; A61P 35/00

(57) ABSTRACT

The disclosure provides novel substituted imidazo [1, 5-b] pyridazine compounds as represented in Formula I: wherein A, R₀, R₁, R₂ and R₃ are defined herein. The compounds of Formula I are kinase inhibitors, especially ATR kinase inhibitors. Therefore, the compounds of the disclosure may be used to treat ATR-mediated diseases, disorders and conditions, such as cancer.

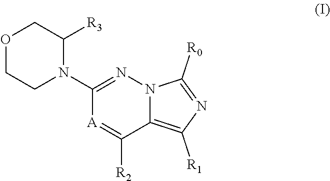

### TECHNICAL FIELD

This disclosure is in the field of medicinal chemistry. In particular, the disclosure relates to substituted imidazo[1,5-b]pyridazine compounds, and the use of these compounds as therapeutically effective kinase inhibitors and anti cancer drugs.

### BACKGROUND

Ataxia telangiectasia and Rad3-related kinase (ATR) is a protein kinase that responds to cells involved in DNA damage. Activated ATR can regulate cell life process through various signals, including interruption of cell cycle, inhibition of replication origin, initiation of replication fork, repair of DNA double strands, etc (Enriquez-Rios V, et al, 2017). ATR kinase regulates cell response to DNA damage, which is usually called DNA damage response (DDR), by acting together with ATM (ataxia telangiectasia mutated) kinase and many other proteins. When a cell recognizes DNA damage through DDR, it will immediately initiate the DNA repair process, activate the cell cycle checkpoint, and hinder the process of normal cell cycle, thereby providing time for DNA repair. Without DDR, cells are more sensitive to endogenous cell damage or DNA damage caused by chemotherapy and radiotherapy for treating cancer, and are more likely to die.

Healthy cells can rely on different proteins for DNA repair, including ATM, ATR kinase in DDR, etc. Under normal circumstances, these proteins can repair DNA by regulating downstream regulatory factors. However, many cancer cells have defects in DNA repair pathway, therefore they are more dependent on the remaining intact DNA repair proteins, including AIR. ATR is a key member of DDR that responds to damaged DNA replication, and is crucial to maintain the stability and integrity of a genome and improve cell survival. When intracellular DNA damage occurs, ATR is recruited to the site of DNA damage, which in turn results in various proteins participating in the regulation of AIR activation. Activated ATR regulates some important cellular processes. Many cancer cells lack key tumor suppressor genes, which can cause cancer cells more dependent on ATR pathway than normal cells to regulate DNA damage repair and improve cell survival, making ATR a promising target for cancer treatment.

ATR inhibitors can be used alone or in combination with DNA damaging agents for cancer treatment, since they block the DNA replication mechanism, which is more important for cell survival in many cancer cells than healthy normal cells. In fact, ATR inhibitors have been shown to be effective as single active agents for cancer cells and as sensitizers for radiotherapy and chemotherapy. At the same time, ATR inhibitors can also be used in combination with other DDR-related targeted drugs, such as PARP inhibitors.

Various AIR kinase inhibitors have been disclosed. For example, WO2011154737 disclosed morpholino pyrimidine compounds as AIR kinase inhibitors; WO2016020320 disclosed 2-(morpholin-4-yl)-1,7-naphthyridine compounds as ATR kinase inhibitors; WO2020049017 disclosed 5-morpholin-4-yl-pyrazolo[4,3-b]pyridine derivatives as AIR kinase inhibitors; WO2020087170 disclosed substituted fused heteroaryl compounds as AIR kinase inhibitors; WO2020259601 disclosed substituted imidazopyridazine compounds as AIR kinase inhibitors; WO2021098811 disclosed pyrazoloheteroaryl derivatives as ATR kinase inhibitors; CN112851668 disclosed a series of compounds as ATR kinase inhibitors; and CN113135942 disclosed condensed pyrimidine derivatives as ATR kinase inhibitors.

SUMMARY OF THE DISCLOSURE

The disclosure provides substituted imidazo[1,5-b]pyridazine compounds as represented in Formula I (including Formulae II, III and IV), the compounds can be used as kinase inhibitors.

The disclosure also provides pharmaceutical compositions comprising an effective amount of the compound of Formula I (including Formulae II, III and IV) for the treatment of cancer.

In a specific embodiment, the pharmaceutical composition may also contain one or more pharmaceutically acceptable carriers or diluents, for the treatment of cancer.

In a specific embodiment, the pharmaceutical composition may also contain at least one known anticancer drug or pharmaceutically acceptable salts thereof, for the treatment of cancer.

The disclosure is also directed to methods for the preparation of novel compounds of Formula I (including Formulae II, III and IV).

### DETAILED DESCRIPTION OF THE DISCLOSURE

It should be understood that the characteristics of the embodiments described herein can be arbitrarily combined to form the technical solution of this disclosure. The definition of each group herein can apply to any of the embodiments described herein. For example, the definitions of the substituents of alkyl herein apply to any of the embodiments described herein unless the substituents of alkyl are clearly defined in the embodiment.

Specifically, the disclosure provides compounds represented by Formula I:

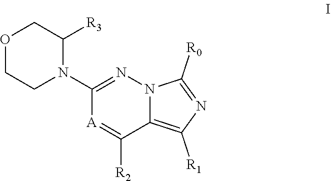

or stereoisomers, tautomers, N-oxides, hydrates, solvates, isotope-substituted derivatives, or pharmaceutically acceptable salts thereof or mixtures thereof, or prodrugs thereof, wherein:
A is N or CH;
R₀ is an optionally substituted aryl, an optionally substituted heterocyclic group, an optionally substituted carbocyclic group, an optionally substituted heteroaryl, an optionally heteroaryl alkyl,

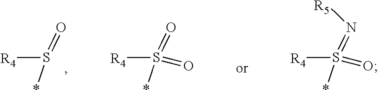

wherein * indicates an attachment position of the group to the rest of the compound;
R₁ is halogen, an optionally substituted C₁-C₆ alkyl, an optionally substituted C₃-C₆ cycloalkyl, an optionally substituted C₂-C₆ alkenyl, or an optionally substituted C₂-C₆ alkynyl;
R₂ is halogen, C₁-C₆ alkyl, C₁-C₆ alkoxy, C₂-C₆ alkenyl, C₂-C₆ alkynyl, carbocyclic group, heterocyclic group, aryl, heteroaryl, —(SO)R₄, —(SO₂)R₄, —SR₄, —NR₆R_{7,} —(CO)R₆, —(CO)OR₆, —(CO)NR₆R₇, —(SO₂)NR₆R₇, —NR₆(SO₂)R₄, —((SO)═NR₅)R₈, —N═(SO)—R₄)R₈, —SiR₅R₈R₉, —(PO)(OR₆)₂, —(PO)(OR₆)R₈ or —(PO)(R₈)₂, wherein the said C₁-C₆ alkyl, C₁-C₆ alkoxy, C₃-C₆ cycloalkyl, C₂-Co alkenyl, C₂-C₆ alkynyl, carbocyclic group, heterocyclic group, aryl and heteroaryl each are optionally substituted;
R₃ is hydrogen or an optionally substituted C₁-C₆ alkyl;
R₄ is an optionally substituted alkyl or an optionally substituted alkylaryl, preferably the aryl is phenyl;
R₅ is hydrogen, an optionally substituted alkyl, —(CO)OR₆ or —(CO)NR₆R₇,
R₆ and R₇ are independently hydrogen, an optionally substituted C₁-C₁₀ alkyl, an optionally substituted carbocyclic group, an optionally substituted heterocyclic group, an optionally substituted aryl or an optionally substituted heteroaryl; or R₆ and R₇ together with the N and C to which they are attached form an optionally substituted 4-7 membered cyclic amino group, which optionally comprises one or more additional heteroatoms selected from O, N and S;
R₈ is C₁-C₄ alkyl, or in the case of —N═(SO)R₄R₈, R₄ and R₈ together with the S to which they attached form a 5-8 membered heterocycloalkyl; and
R₉ is hydrogen or C₁-C₄ alkyl.

Preferably, in the definition of the above groups of Formula I, unless specified, the said carbocyclic group preferably contains 3-8 carbon atoms in the ring, such as C₃-C₈ cycloalkyl; the said aryl is preferably 6-14 membered aryl; the said heteroaryl is preferably 5-10 membered heteroaryl, and the said heterocyclic group is preferably 4-9 membered heterocyclic group.

In one or more of the foregoing embodiments of the compound of Formula I, A is CH.

in one or more of the foregoing embodiments of the compound of Formula I, R₀ is an optionally substituted alkylsulfonyl, an optionally substituted aryl, an optionally substituted heterocyclic group, an optionally substituted carbocyclic group or an optionally substituted heteroaryl. Preferably, the heteroaryl is a 5- or 6-membered heteroaryl containing at least one nitrogen atom, preferably a 5-membered heteroaryl containing two nitrogen atoms. Preferably, the substituents of the alkylsulfonyl, heterocyclic group, carbocyclic group, aryl and heteroaryl may be selected from a group consisting of C₁-C₄ alkyl, halogen, hydroxy, C₁-C₄ alkoxy and amino. Preferably, the number of substituents inn Ru may be 1-3. More preferably, R₀ is sulfonyl substituted with C₁-C₄ alkyl; or pyrazolyl, pyrazolyl, or imidazolyl optionally substituted with 1 or 2 substituents selected from a group consisting of C₁-C₄ alkyl, halogen, hydroxyl, C₁-C₄ alkoxy and amino. In some embodiments, R₀ is unsubstituted pyrazolyl, unsubstituted pyrrolyl, unsubstituted imidazolyl, or pyrazolyl substituted with one C₁-C₄ alkyl. In some embodiments, R₀ is 1H-pyrazol-5-yl optionally substituted with one C₁-C₄ alkyl.

In one or more of the foregoing embodiments of the compound of Formula I, R₁ is halogen, C₁-C₆ alkyl, C₃-C₆ cycloalkyl or C₂-C₆ alkenyl optionally substituted with 1-6 substituents selected from halogen, hydroxyl and —NRₐR_{b}, wherein the said Rₐ and Rₕ are independently hydrogen or C₁-C₄ alkyl. Preferably, R₁ is halogen, C₁-C₄ alkyl, C₃-C₄ cycloalkyl or C₂-C₄ alkenyl. More preferably, R₁ is halogen, C₁-C₃ alkyl, C₃-C₄ cycloalkyl or C₂-C₃ alkenyl.

In one or more of the foregoing embodiments of the compound of Formula I, R₂ is carbocyclic group, heterocyclic group, aryl, or heteroaryl, wherein the said carbocyclic group, heterocyclic group, aryl and heteroaryl each are optionally substituted. Preferably, R₂ is an optionally substituted aryl, an optionally substituted heterocyclic group or an optionally substituted heteroaryl. Preferably, the said aryl is phenyl or naphthyl. Preferably, the said heterocyclic group is a 4-7 membered heterocyclic group containing N and/or O. Preferably, the said heterocyclic group selected from tetrahydropyranyl, tetrahydrofuranyl, oxetanyl, azetidinyl, pyrrolidinyl, piperidinyl and piperazinyl. Preferably, the said heteroaryl is a 5 or 6 membered heteroaryl containing N. Preferably, the said heteroaryl is selected from a group consisting of pyrazolyl, imidazolyl, pyridyl, pyrimidinyl, pyrrolyl and triazolyl. Preferably, R₂ is an optionally substituted phenyl, an optionally substituted pyrazotyl, an optionally substituted pyridyl, or an optionally substituted tetrahydropyranyl. Preferably, the substituent of R₂ is selected from C₁-C₆ alkyl, haloC₁-C₆ alkyl, cyano, halogen and alkylsulfonyl (such as sulfonyl substituted with C₁-C₄ alkyl). The number of substituents can be 1-3. In some preferred embodiments, R₂ is phenyl optionally substituted with 1 or 2 substituents selected from C₁-C₆ alkyl, halogen, cyano and sulfonyl substituted with C₁-C₄ alkyl; pyrazolyl or pyridyl optionally substituted with 1 or 2 substituents selected from C₁-C₆ alkyl, haloC₁-C₆ alkyl and halogen; or tetrahydropyranyl optionally substituted with 1 or 2 substituents selected from C₁-C₆ alkyl, haloC₁-C₆ alkyl and halogen.

Preferably, R₂ is selected from:

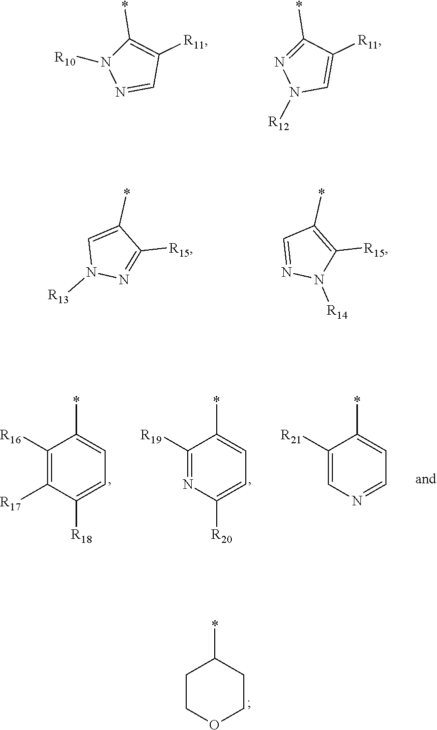

wherein, R₁₀ is hydrogen, C₁-C₃ alkyl or haloC₁-C₃ alkyl; each R₁₁ is independently hydrogen or C₁-C₃ alkyl; R₁₂ is hydrogen or halo C₁-C₃ alkyl; R₁₃ is hydrogen or C₁-C₃ alkyl; R₁₄ is hydrogen or C₁-C₃ alkyl; each R₁₅ is independently hydrogen or C₁-C₃ alkyl, preferably C₁-C₃ alkyl; R₁₆ is hydrogen, halogen or C₁-C₃ alkyl, preferably halogen or C₁-C₃ alkyl; R₁₇ is hydrogen or cyano; R₁₈ is hydrogen or C₁-C₃ alkyl substituted sulfonyl; R₁₉ is hydrogen, halogen, C₁-C₃ alkyl or halo C₁-C₃ alkyl; R₂₀ is hydrogen or C₁-C₃ alkyl; R₂₁ is halogen, C₁-C₃ alkyl or halo C₁-C₃ alkyl, preferably halogen; wherein, * refers to an attachment position of R₂ to the rest of the compound.

More preferably, R₂ is selected from

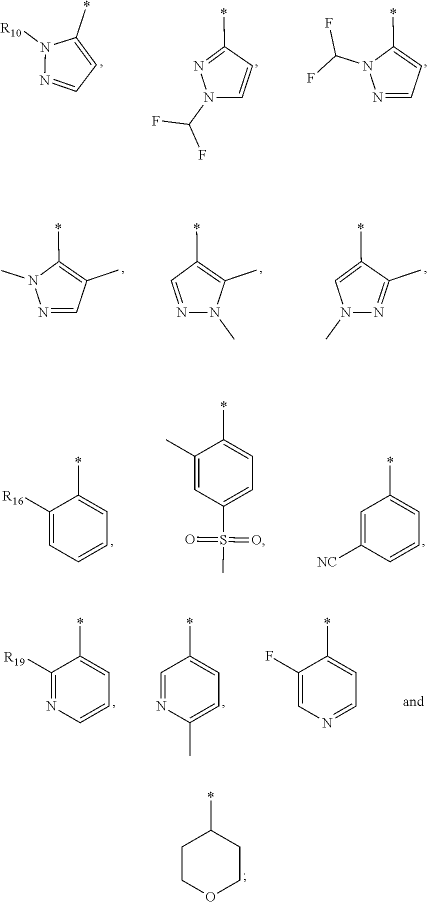

wherein, R₁₀ is C₁-C₃ alkyl, such as methyl, ethyl, and isopropyl; R₁₆ is methyl or fluoro; R₁₉ is methyl, fluoro or trifluoromethyl; wherein * refer to an attachment position of R₂ to the rest of the compound.

More preferably, R₂ is:

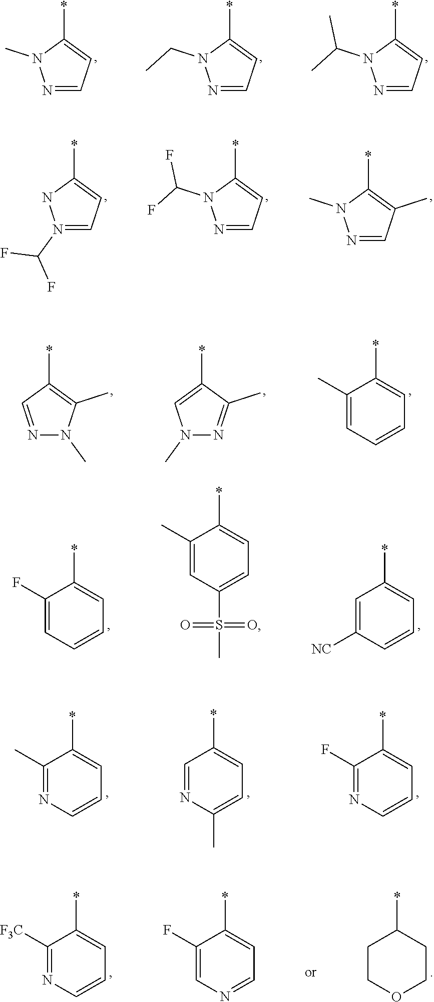

in one or more of the foregoing embodiments of the compound of Formula I, R₃ is a C₁-C₆ alkyl optionally substituted with 1-6 substituents selected from a group consisting of halogen, hydroxyl, —NRₐR_{b} and halo C₁-C₄ alkyl, wherein Rₐ and R_{b}are independently hydrogen or C₁-C₄ alkyl. Preferably, R₃ is C₁-C₄ alkyl ; such as methyl. Preferably, R₃ is in an R-configuration.

in one or more of the foregoing embodiments of the compound of Formula I, R₄ is alkyl or alkylaryl, which is optionally substituted with 1-6 substituents selected from halogen, hydroxyl and —NRₐR_{b}, wherein Rₐ and R_{b} are independently hydrogen or C₁-C₄ alkyl. In some embodiments, R₄ is an optionally substituted C₁-C₄ alkyl or an optionally substituted C₁-C₄ alkylaryl (preferably the aryl is phenyl), which is optionally substituted with 1-6 substituents selected from halogen, hydroxyl and —NRₐR_{b}, wherein Rₐ and R_{b} are independently hydrogen or C₁-C₄ alkyl.

In each of the groups described in R₅, R₆ and R₇, when substituted, the substituent may be selected from halogen, hydroxyl, C₁-C₄ alkyl, —NRₐR_{b}, halo C₁-C₄ alkyl, and C₃-C₈ cycloalkyl optionally substituted by 1 or 2 substituents selected from C₁-C₄ alkyl, halogen, hydroxyl, —NRₐR_{b} and halo C₁-C₄ alkyl, wherein Rₐ and R_{b} are independently hydrogen or C₁-C₄ alkyl; the number of substituents can be 1-6. In some embodiments, R₅ is H or C₁-C₄ alkyl optionally substituted with 1-6 substituents selected from halogen, hydroxyl and —NRₐR_{b}, wherein Rₐ and Rₕ are independently hydrogen or C₁-C₄ alkyl. In some embodiments, R₆ and R₇ are each independently hydrogen, an optionally substituted C₁-C₄ alkyl, an optionally substituted C₃-C₆ cycloalkyl, an optionally substituted aryl or an optionally substituted heteroaryl; or R₆ and R₇ together to which they are attached form an optionally substituted 4-7 membered cyclic amino group, which optionally comprises one or more additional heteroatoms selected from O, N and S. In some embodiments, R₈ is C₁-C₄ alkyl, or in the case of —N═(SO)R₄R₈, R₄ and R₈ together to which they attached form a 5-S membered heterocycloalkyl.

The disclosure provides compounds represented by Formula II:

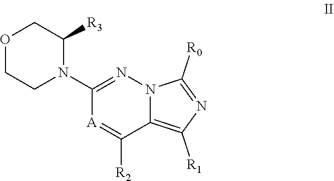

or stereoisomers, tautomers, N-oxides, hydrates, solvates, isotope-substituted derivatives, or pharmaceutically acceptable salts thereof or mixtures thereof, or prodrugs thereof, wherein:

A, R₀, R₁, R₂ and R₃ are as defined in any one of the embodiments of Formula I as described above.

In one or more of the foregoing embodiments of the compound of Formula II, A is CH.

In one or more of the foregoing embodiments of the compound of Formula II, R₀ is an optionally substituted alkylsulfonyl, an optionally substituted aryl, an optionally substituted heterocyclic group, an optionally substituted carbocyclic group or an optionally substituted heteroaryl. Preferably, the heteroaryl is a 5- or 6-membered heteroaryl containing at least one nitrogen atom, preferably a 5-membered heteroaryl containing two nitrogen atoms. Preferably, the substituents of the alkylsulfonyl, heterocyclic group, carbocyclic group, aryl and heteroaryl may be selected from a group consisting of C₁-C₄ alkyl, halogen, hydroxy, C₁-C₄ alkoxy and amino. Preferably, the number of substituents on R₀ may be 1-3. More preferably, R₀ is sulfonyl substituted with C₁-C₄ alkyl; or pyrazolyl, pyrazolyl, or imidazolyl optionally substituted with 1 or 2 substituents selected from a group consisting of C₁-C₄ alkyl, halogen, hydroxyl, C₁-C₄ alkoxy and amino. In some embodiments, R₀ is unsubstituted pyrazolyl, unsubstituted pyrrolyl, or unsubstituted imidazolyl, or pyrazolyl substituted with one C₁-C₄ alkyl, in some embodiments, R₀ is 1H-pyrazol-5-yl optionally substituted with one C₁-C₄ alkyl.

In one or more of the foregoing embodiments of the compound of Formula II, R₁ is halogen, C₁-C₆ alkyl, C₃-C₇ cycloalkyl or C₂-C₆ alkenyl optionally substituted with 1-6 substituents selected from halogen, hydroxyl and —NRₐR_{b}, wherein the said Rₐ and R_{b} are independently hydrogen or C₁-C₄ alkyl. Preferably, R₁ is halogen, C₁-C₄ alkyl, C₁-C₄ cycloalkyl or C₂-C₄ alkenyl. More preferably, RI is halogen, C₁-C₃ alkyl or C₂-C₃ alkenyl,

In one or more of the foregoing embodiments of the compound of Formula II, R₂ is carbocyclic group, heterocyclic group, aryl, or heteroaryl, wherein the said carbocyclic group, heterocyclic group, aryl and heteroaryl each are optionally substituted. Preferably, R₂ is an optionally substituted aryl, an optionally substituted heterocyclic group or an optionally substituted heteroaryl. Preferably, the said aryl is phenyl or naphthyl. Preferably, the said heterocyclic group is a 4-7 membered heterocyclic group containing N and/or O. Preferably, the said heterocyclic group selected from tetrahydropyranyl, tetrahydrofuranyl, oxetanyl, azetidinyl, pyrrolidinyl, piperidinyl and piperazinyl. Preferably, the said heteroaryl is a 5 or 6 membered heteroaryl containing N. Preferably, the said heteroaryl is selected from a group consisting of pyrazolyl, imidazolyl, pyridyl, pyrimidinyl, pyrrolyl and triazolyl. Preferably, R₂ is an optionally substituted phenyl, an optionally substituted pyrazolyl, an optionally substituted pyridyl, or an optionally substituted tetrahydropyranyl. Preferably, the substituent of R₂ is selected from C₁-C₆ alkyl, haloC₁-C₆ alkyl, cyano, halogen and alkylsulfonyl (such as sulfonyl substituted with C₁-C₄ alkyl). The number of substituents can be 1-3. In some preferred embodiments, R₂ is phenyl optionally substituted with 1 or 2 substituents selected from C₁-C₆ alkyl, halogen, cyano and sulfonyl substituted with C₁-C₄ alkyl; pyrazolyl or pyridyl optionally substituted with 1 or 2 substituents selected from C₁-Cu alkyl, halo C₁-C₆ alkyl and halogen; or tetrahydropyranyl optionally substituted with 1 or 2 substituents selected from C₁-C₆ alkyl, halo C₁-C₆ alkyl and halogen.

Preferably, R₂ is selected from:

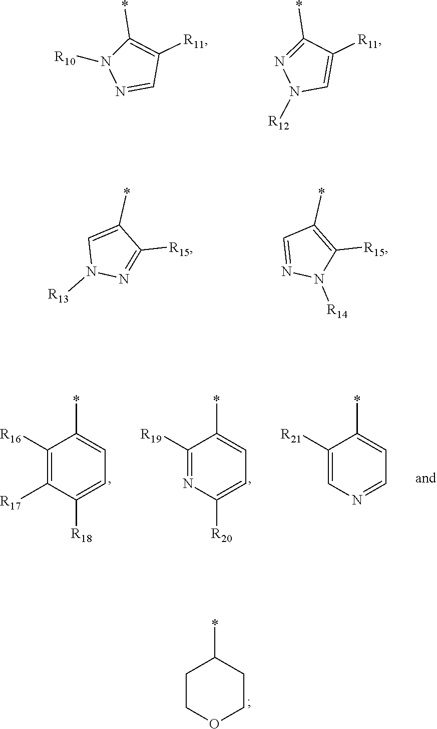

wherein, R₁₀ is hydrogen, C₁-C₃ alkyl or haloC₁-C₃ alkyl; each is independently hydrogen or C₁-C₃ alkyl; R₁₂ is hydrogen or halo C₁-C₃ alkyl; R₁₃ is hydrogen or C₁-C₃ alkyl; R₁₄ is hydrogen or C₁-C₃ alkyl; each R₁₅ is independently hydrogen or C₁-C₃ alkyl, preferably C₁-C₃ alkyl; R₁₆ is hydrogen, halogen or C₁-C₃ alkyl, preferably halogen or C₁-C₃ alkyl; R₁₇ is hydrogen or cyano; R₁₈ is hydrogen or C₁-C₃ alkyl substituted sulfonyl; R₁₉ is hydrogen, halogen, C₁-C₃ alkyl or halo C₁-C₃ alkyl; R₂₀ is hydrogen or C₁-C₃ alkyl; R₁ is halogen, C₁-C₃ alkyl or halo C₁-C₃ alkyl, preferably halogen; wherein, * refers to an attachment position of R₂ to the rest of the compound.

More preferably, R₂ is selected from

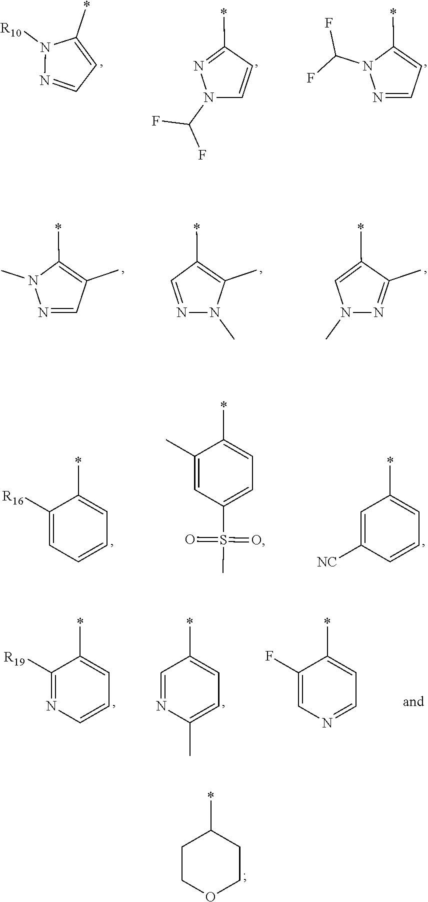

wherein, R₁₀ is C₁-C₁ alkyl, such as methyl, ethyl, and isopropyl; R₁₆ is methyl or fluoro; R₁₉ is methyl, fluoro or trifluoromethyl; wherein * refer to an attachment position of R₂ to the rest of the compound.

More preferably, R₂ is:

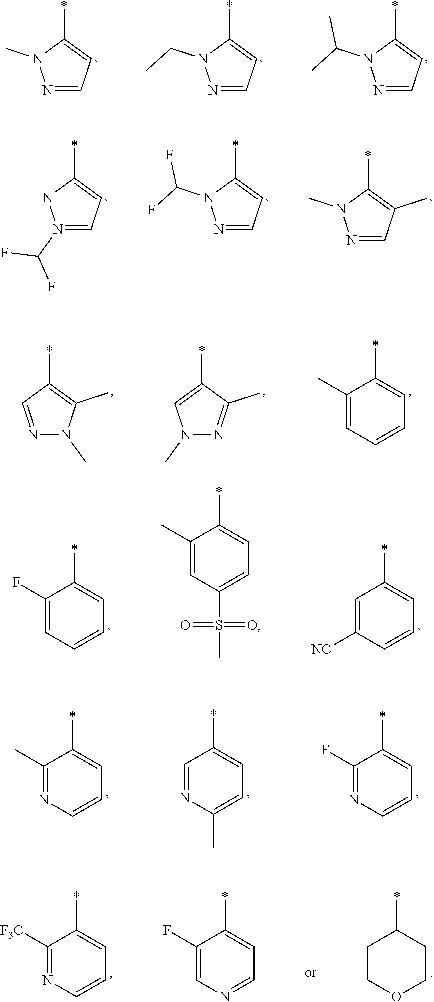

In one or more of the foregoing embodiments of the compound of Formula I, R₃ is a C₁-C₆ alkyl optionally substituted with 1-6 substituents selected from a group consisting of halogen, hydroxyl, —NRₐR_{b} and halo C₁-C₄ alkyl, wherein Rₐ and R_{b} are independently hydrogen or C₁-C₄ alkyl. Preferably, R₃ is C₁-C₄ alkyl, such as methyl.

In one or more of the foregoing embodiments of the compound of Formula II, R₄ is alkyl or alkylaryl, which is optionally substituted with 1-6 substituents selected from halogen, hydroxyl and —NRₐR_{b}, wherein Rₐ and R_{b} are independently hydrogen or C₁-C₄ alkyl. In some embodiments, R₄ is an optionally substituted C₁-C₄ alkyl or an optionally substituted C₁-C₄ alkylaryl (preferably the aryl is phenyl), which is optionally substituted with 1-6 substituents selected from halogen, hydroxyl and —NRₐR_{b}, wherein Rₐ and R_{b} are independently hydrogen or C₁-C₄ alkyl.

In each of the groups described in R_{5,} R₆ and R₇ of the compounds of Formula II, when substituted, the substituent may be selected from halogen, hydroxyl, C₁-C₄ alkyl, halo C₁-C₄ alkyl, C₃-C₈ cycloalkyl which optionally 1 or 2 substituents selected from C₁-C₄ alkyl, halogen, hydroxyl, —NRₐR_{b} and halo C₁-C₄ alkyl; the number of substituents can be 1-6. In some embodiments, R₅ is H or a C₁-C₄ alkyl optionally substituted with 1-6 substituents selected from halogen, hydroxyl and —NRₐR_{b}, wherein Rₐ and R_{b} are independently hydrogen or C₁-C₄ alkyl. In some embodiments, R₆ and R₇ are each independently hydrogen, an optionally substituted C₁-C₄ alkyl, an optionally substituted C₃-C₆ cycloalkyl, an optionally substituted aryl or an optionally substituted heteroaryl; or R₆ and R₇ together to which they are attached form an optionally substituted 4-7 membered cyclic amino group, which optionally comprises one or more additional heteroatoms selected from O, N and S. In some embodiments, R₈ is C₁-C₄ alkyl, or in the case of —N═(SO)R₄R₈, R₄ and R₈ together to which they attached form a 5-8 membered heterocycloalkyl.

The disclosure provides compounds represented by Formula III:

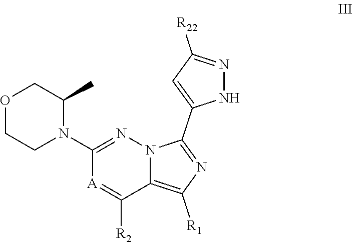

or stereoisomers, tautomers, N-oxides, hydrates, solvates, isotope-substituted derivatives, or pharmaceutically acceptable salts thereof, or mixtures thereof, or prodrugs thereof, wherein:
A, R₁ and R₂ are as defined in any one of the embodiments of Formulae I and II as described above;
R₂₂ is hydrogen, halo or an optionally substituted C₁-C₆ alkyl.

In one or more of the foregoing embodiments of the compound of Formula III, A is CH.

In one or more of the foregoing embodiments of the compound of Formula III, R₂₂ is H or C₁-C₆ alkyl optionally substituted with 1-6 substituents selected from halogen, hydroxyl and —NₐR_{b}, wherein the said Rₐ and R_{b} are independently hydrogen or C₁-C₄ alkyl. Preferably, R₂₂ is hydrogen or C₁-C₃ alkyl. In some embodiments, R₂₂ is H.

In one or more of the foregoing embodiments of the compound of Formula III, R₁ is halogen, C₁-C₆ alkyl, C₃-C₆ cycloalkyl or C₂-C₆ alkenyl optionally substituted with 1-6 substituents selected from halogen, hydroxyl and —NRₐR_{b}, wherein the said Rₐ and R_{b} are independently hydrogen or C₁-C₄ alkyl. Preferably, R₁ is halogen, C₁-C₄ alkyl, C₃-C₄ cycloalkyl or C₂-C₄ alkenyl. More preferably, R₁ is halogen, C₁-C₃ alkyl or C₂-C₃ alkenyl.

In one or more of the foregoing embodiments of the compound of Formula I, R₂ is carbocyclic group, heterocyclic group, aryl, or heteroaryl, wherein the said carbocyclic group, heterocyclic group, aryl and heteroaryl each are optionally substituted. Preferably, R₂ is an optionally substituted aryl, an optionally substituted heterocyclic group or an optionally substituted heteroaryl. Preferably, the said aryl is phenyl or naphthyl. Preferably, the said heterocyclic group is a 4-7 membered heterocyclic group containing N and/or O. Preferably, the said heterocyclic group selected from tetrahydropyranyl, tetrahydrofuranyl, oxetanyl, azetidinyl, pyrrolidinyl, piperidinyl and piperazinyl. Preferably, the said heteroaryl is a 5 or 6 membered heteroaryl containing N. Preferably, the said heteroaryl is selected from a group consisting of pyrazolyl, imidazolyl, pyridyl, pyrimidinyl, pyrrolyl and triazolyl. Preferably, R₂ is an optionally substituted phenyl, an optionally substituted pyrazolyl, an optionally substituted pyridyl, or an optionally substituted tetrahydropyranyl. Preferably, the substituent of R₂ is selected from C₁-C₆ alkyl, haloC₁-C₆ alkyl, cyano, halogen and alkyl sulfonyl (such as sulfonyl substituted with C₁-C₄ alkyl). The number of substituents can be 1-3. In some preferred embodiments, R₂ is phenyl optionally substituted with 1 or 2 substituents selected from C₁-C₆ alkyl, halogen, cyano and sulfonyl substituted with C₁-C₄ alkyl; pyrazolyl or pyridyl optionally substituted with 1 or 2 substituents selected from C₁-C₆ alkyl, haloC₁-C₆ alkyl and halogen; or tetrahydropyranyl optionally substituted with 1 or 2 substituents selected from C₁-C₆ alkyl, haloC₁-C₆ alkyl and halogen.

Preferably, R₂ is selected from:

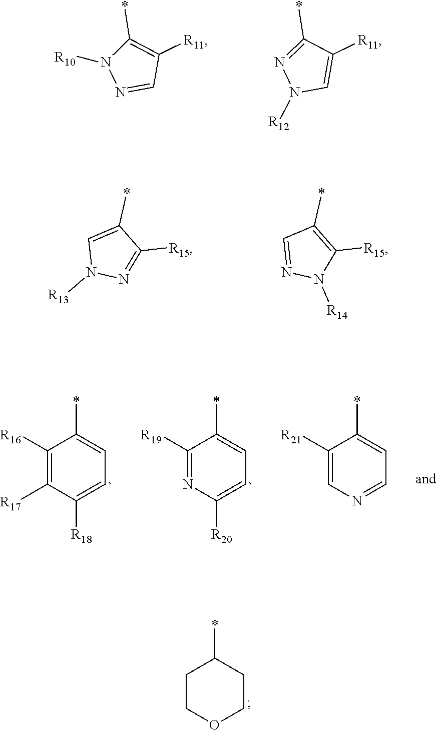

wherein, R₁₀ is hydrogen, C₁-C₃ alkyl or haloC₁-C₃ alkyl; each R₁₁ is independently hydrogen or C₁-C₃ alkyl; R₁₂ is hydrogen or halo C₁-C₃ alkyl; R₁₃ is hydrogen or C₁-C₃ alkyl; R₁₄ is hydrogen or C₁-C₃ alkyl; each R₁₅ is independently hydrogen or C₁-C₃ alkyl, preferably C₁-C₃ alkyl; R₁₆ is hydrogen, halogen or C₁-C₃ alkyl, preferably halogen or C₁-C₃ alkyl; R₁₇ is hydrogen or cyano; R₁₈ is hydrogen or C₁-C₃ alkyl substituted sulfonyl; R₁₉ is hydrogen, halogen, C₁-C₃ alkyl or halo C₁-C₃ alkyl; R₂₀ is hydrogen or C₁-C₃ alkyl; R₂₁ is halogen, C₁-C₃ alkyl or halo C₁-C₃ alkyl, preferably halogen; wherein, * refers to an attachment position of R₂ to the rest of the compound.

More preferably, R₂ is selected from

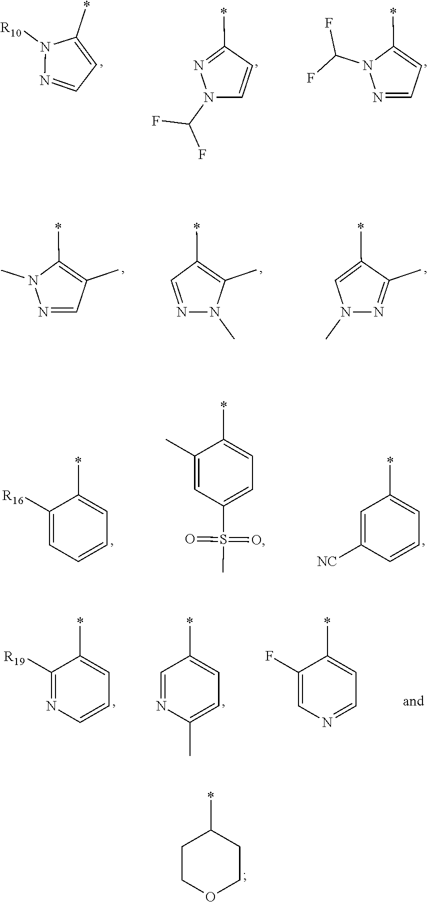

wherein, R₁₀ is C₁-C₃ alkyl, such as methyl, ethyl, and isopropyl; R₁₆ is methyl or fluoro; R₁₉ is methyl, fluoro or trifluoromethyl; wherein * refer to an attachment position of R₂ to the rest of the compound.

More preferably, R₂ is:

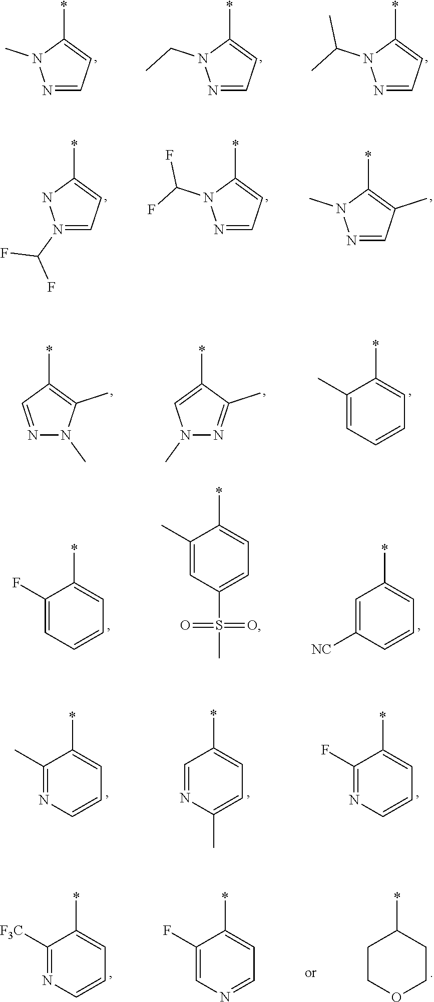

The disclosure provides compounds represented by Formula IV:

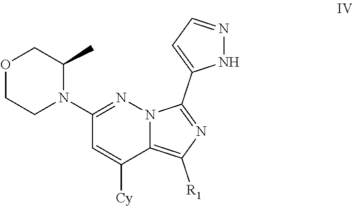

or stereoisomers, tautomers, N-oxides, hydrates, solvates, isotope-substituted. derivatives, or pharmaceutically acceptable salts thereof, or mixtures thereof, or prodrugs thereof, wherein:

R₁ is as defined in any one of the embodiments of Formulae I, II and III as described above;
Cy is carbocyclic group, heterocyclic group, aryl, heteroaryl, —NR₆R₇, —NR₆(SO₂)R₄, or —N=(SO)R₄R₈, wherein the said carbocyclic group, heterocyclic group, aryl and heteroaryl can be optionally substituted; wherein, R₄ is an optionally substituted alkyl or an optionally substituted alkylaryl (preferably the aryl is phenyl); R₆ and R₇ are each independently hydrogen, an optionally substituted C₁-C₁₀ alkyl, an optionally substituted cycloalkyl, an optionally substituted aryl or an optionally substituted heteroaryl; or R₆ and R₇ together to which they are attached form an optionally substituted 4-7 membered cyclic amino group, which optionally comprises one or more additional heteroatoms selected from O, N and S; R₈ is C₁-C₄ alkyl, or in the case of —N═(SO)R₄R₈, R₄ and R₈ together to which they attached form a 5-8 membered heterocycloalkyl,

In one or more of the embodiments of the compound of Formula IV, when R₁ is substituted, the substituent may be selected from 1-6 groups of halogen, hydroxyl and —NRₐR_{b}, wherein Rₐ and R_{b} are each independently hydrogen or C₁-C₄ alkyl. Preferably, R₁ is halogen, C₁-C₄ alkyl, C₃-C₄ cycloalkyl or C₂-C₄ alkenyl. More preferably, R₁ is halogen, C₁-C₃ alkyl or C₂-C₃ alkenyl.

in one or more of the embodiments of the compound of Formula IV, Cy is carbocyclic group, heterocyclic group, aryl, or heteroaryl, wherein the carbocyclic group, heterocyclic group, aryl and heteroaryl can be optionally substituted. Preferably, Cy is an optionally substituted aryl, an optionally substituted heterocyclic group or an optionally substituted heteroaryl. Preferably, the said aryl is phenyl or naphthyl. Preferably, the said heterocyclic group is a 4-7 membered heterocyclic group containing N and/or O. Preferably, the said heterocyclic group selected from tetrahydropyranyl, tetrahydrofuranyl, oxetanyl, azetidinyl, pyrrolidinyl, piperidinyl and piperazinyl. Preferably, the said heteroaryl is a 5 or 6 membered heteroaryl containing N. Preferably, the said heteroaryl is selected from a group consisting of pyrazolyl, imidazolyl, pyridyl, pyrimidinyl, pyrrolyl and triazolyl. Preferably, Cy is an optionally substituted phenyl, an optionally substituted pyrazolyl, an optionally substituted pyridyl, an optionally substituted tetrahydropyranyl. Preferably, the substituent of Cy is selected from C₁-C₆ alkyl, halo C₁-C₆ alkyl, cyano, halogen and alkyl sulfonyl (such as sulfonyl substituted with C₁-C₄ alkyl), The number of substituents can be 1-3. In some preferred embodiments, Cy is phenyl optionally substituted with 1 or 2 substituents selected from C₁-C₆ alkyl, halogen, cyano and sulfonyl substituted with C₁-C₄ alkyl; pyrazolyl or pyridyl optionally substituted with 1 or 2 substituents selected from C₁-C₆ alkyl, halo C₁-C₆ alkyl and halogen; or tetrahydropyranyl optionally substituted with 1 or 2 substituents selected from C₁-C₆ alkyl, halo C₁-C₆ alkyl and halogen. In some embodiments, Cy is pyrazolyl optionally substituted with C₁-C₆ alkyl, and preferably, one or two ring N atoms of the pyrazolyl group are substituted.

In one or more of the embodiments of the compound of Formula IV, Cy is selected from a group consisting of:

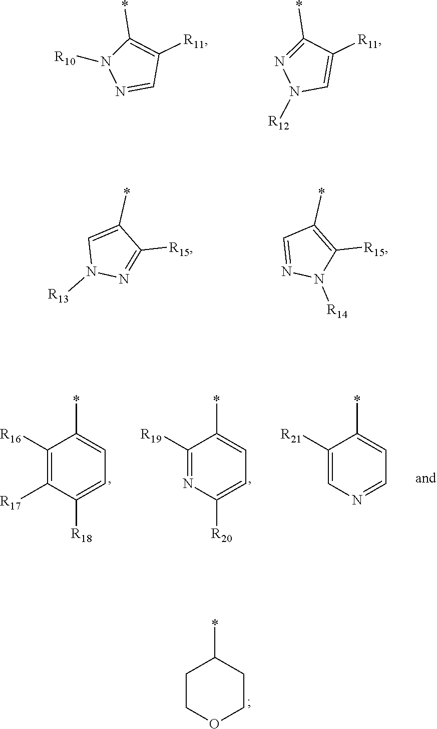

wherein, R₁₀ is hydrogen, C₁-C₃ alkyl or haloC₁ -C₃ alkyl; each R₁₁ is independently hydrogen or C₁-C₃ alkyl; R₁₂ is hydrogen or halo C₁-C₃ alkyl; R₁₃ is hydrogen or C₁-C₃ alkyl; R₁₄ is hydrogen or C₁-C₃ alkyl; each R₁₅ is independently hydrogen or C₁-C₃ alkyl, preferably C₁-C₃ alkyl; R₁₆ is hydrogen, halogen or C₁-C₃ alkyl, preferably halogen or C₁-C₃ alkyl; R₁₇ is hydrogen or cyano; R₁₈ is hydrogen or C₁-C₃ alkyl substituted sulfonyl; R₁₉ is hydrogen, halogen, C₁-C₃ alkyl or halo C₁-C₃ alkyl; R₂₀ is hydrogen or C₁-C₃ alkyl; R₂₁ is halogen, C₁-C₃ alkyl or halo C₁-C₃ alkyl, preferably halogen; wherein, * refers to an attachment position of R₂ to the rest of the compound.

In one or more of the embodiments of the compound of Formula IV, Cy is selected from a group consisting of:

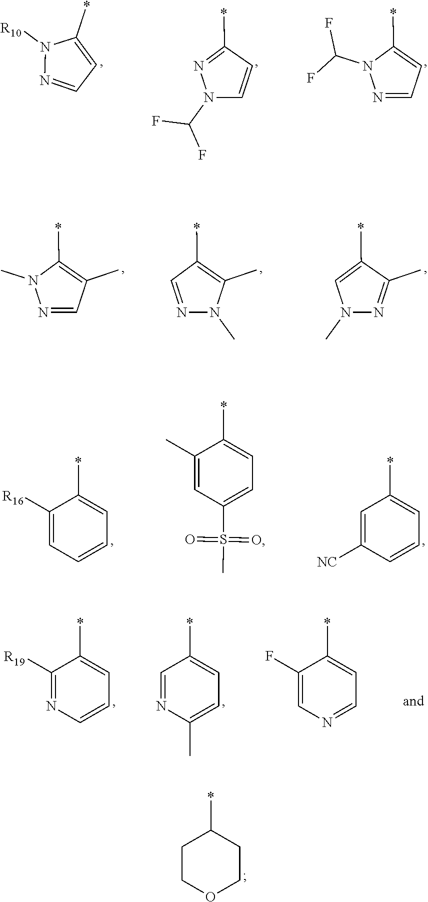

wherein, R₁₀ is C₁-C₃ alkyl, such as methyl, ethyl, and isopropyl; R₁₆ is methyl or fluoro; R₁₉ is methyl, fluoro or trifluoromethyl; wherein * refer to an attachment position of R₂ to the rest of the compound.

In one or more of the embodiments of the compound of Formula IV, Cy is:

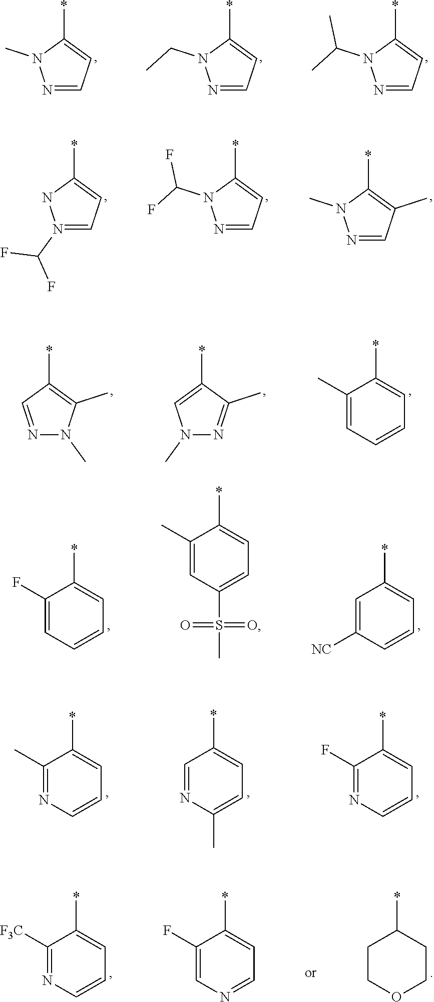

In one or more of the foregoing embodiments, preferred compounds of Formula I (including Formulae II, and IV) include, without limitation:

(R)-3 -methyl -4-(5 -methyl -4-(1 -methyl -1H-pyrazol-5 -yl)-7-(1H-pyrazol-5-yl)imidazo[1,5 b]pyridazin-2-yl)morpholine (Example 1);

(R)-4-(4-(1-ethyl-1H-pyrazol-5-yl)-5-methyl-7-(1H-pyrazol-5-yl)imidazo[1,5-b]pyridazin-2-yl)-3-methylmorpholine (Example 2);

(R)-3-methyl-4-(5-methyl-7-(1H-pyrazol-5-yl)-4-(o-tolyl)imidazo[1,5-b]pyridazin-2-yl)morpholine (Example 3);

(R)-3-methyl-4-(5-methyl-4-(2-methylpyridin-3-yl)-7-(1H-pyrazol-5-yl)imidazo[1,5-b]pyridazin-2-yl)morpholine (Example 4);

(R)-3-methyl-4-(5-methyl-7-(1H-pyrazol-5-yl)-4-(2-(trifluoromethyl)pyridin-3-yl)imidazo[1,5-b]pyridazin-2-yl)morpholine (Example 5);

(R)-3-(5-methyl-2-(3-methylmorpholino)-7-(1H-pyrazol-5-yl)imidazol, [1,5-b]pyridazin-4-yl)benzonitrile (Example 6);

(R)-3-methyl-4-(5-methyl-7-(1H-pyrazol-5-yl)-4-(tetrahydro-2H-pyran-4-yl)imidazol[1,5-b]pyridazin-2-yl)morpholine (Example 7);

(R)-3-methyl-4-(5-methyl-4-(1-methyl-1H-pyrazol-5-yl)-7-(3-methyl-1H-pyrazol-5-yl)imidazo[1,5-b]pyridazin-2-yl)morpholine (Example 8);

(R)-4-(4-(1-isopropyl-1H-pyrazol-5-yl)-5-methyl-7-(1H-pyrazol-5-yl)imidazo[1,5-b]pyridazin-2-yl)-3-methylmorpholine (Example 9);

(R)-4-(4-(1-(difluoromethyl)-1H-pyrazol-3-yl)-5-methyl-7-(1H-pyrazol-5-yl)imidazo[1,5-b]pyridazin-2-yl)-3-methylmorpholine (Example 10);

(R)-4-(4-(1,4-dimethyl-1H-pyrazol -5-yl)-5-methyl-7-(1H-pyrazol-5-yl)imidazo[1,5-b]pyridazin-2-yl)-3-methylmorpholine (Example 11);

(R)-4-(4-(1,5-dimethyl-1H-pyrazol-4-yl)-5-methyl-7-(1H-pyrazol-5-yl)imidazo[1,5-b]pyridazin-2-yl)-3-methylmorpholine (Example 12);

(R)-4-(4-(1,3-dimethyl-1H-pyrazol-4-yl)-5-methyl-7-(1H-pyrazol-5-yl)imidazo[1,5-b]pyridazin-2-yl)-3-methylmorpholine (Example 13);

(R)-4-(4-(2-fluorophenyl)-5-methyl-7-(1H-pyrazol-5-yl)imidazo[1,5-b]pyridazin-2-yl)-3-methylmorpholine (Example 14);

(R)-3-methyl-4-(5-methyl-4-(2-methyl-4-(methylsulfonyl)phenyl)-7-(1H-pyrazol-5-yl)imidazo[1,5-b]pyridazin-2-34)morpholine (Example 15);

(R)-4-(4-(2-fluoropyridin-3-yl)-5-methyl-7-(1H-pyrazol-5-yl)imidazo[1,5-b]pyridazin-2-yl)-3-methylmorpholine (Example 16);

(R)-3-methyl-4-(5-methyl-4-(6-methylpyridin-3yl)-7-(1H-pyrazol-5-yl)imidazo[1,5-b]pyridazin-2-yl)morpholine (Example 17);

(R)-4-(4-(3-fluoropyridin-4-yl)-5-methyl-7-(1H-pyrazol-5-yl)imidazo[1,5-b]pyridazin-2-yl)-3-methylmorpholine (Example 18);

(R)-4-(4-(1-(difluoromethyl)-1H-pyrazol-5-yl)-5-methyl -7-(1H-pyrazol-5-yl)imidazo[1,5-b]pyridazin-2-yl)-3-methylmorpholine (Example 19);

(R)-4-(5-fluoro-4-(1-methyl-1H-pyrazol-5-yl)imidazo[1,5-b]pyridazin-2-yl)-3-methylmorpholine (Example 20);

(R)-4-(5-chloro-4-(1-methyl-1H-pyrazol-5-yl)-7-(1H-pyrazol-5-yl)imidazo[1,5-b]pyridazin-2-yl)-3-methylmorpholine (Example 21);

(R)-4-(5-bromo-4-(1-methyl-1H-pyrazol-5-yl)-7-(1H-pyrazol-5-yl)imidazo[1,5-b]pyridazin-2-yl)-3-methylmorpholine (Example 22);

(R)-3-methyl-4-(4-(1-methyl-1H-pyrazol-5-yl)-7-(1H-pyrazol-5-yl)-5-vinylimidazo[1,5-b]pyridazin-2-yl)morpholine (Example 23);

(R)-4-(5-ethyl-4-(1-methyl-1H-pyrazol-5-yl)-7-(1H-pyrazol-5-yl)imidazo[1,5-b]pyridazin-2-yl)-3-methylmorpholine (Example 24);

(R)-3-Methyl-4-(4-(1-Methyl-1H-pyrazol-5-yl)-5-(prop-1-en-2-yl)-7-(1H-pyrazol-5-yl)imidazo[1,5-b]pyridazin -2-yl)morpholine (Example 25);

(R)-4-(5-isopropyl-4-(1-methyl-1H-pyrazol-5-yl)-7-(1H-pyrazol -5-yl)imidazo[1,5-b]pyridazin-2-yl)-3-methylmorpholine (Example 26);

(R)-4-(5-bromo-4-(1-methyl-1H-pyrazol-5-yl)-7-(1H-pyrazol-5-yl)imidazo[5,1-f][1,2,4]triazin-2-yl)-3-methylmorpholine (Example 28); (R)-3-methyl-4-(5-methyl-4-(1-methyl-1H-pyrazol-5-yl)-7-(1H-pyrazol-5-yl)imidazo[5,1-f][1,2,4]triazin-2-yl)morpholine (Example 29);

or stereoisomers, tautomers, N-oxides, hydrates, solvates, isotope-substituted derivatives, or pharmaceutically acceptable salts thereof, or mixtures thereof, or prodrugs thereof.

The term “hydrogen (H)” as employed herein includes its isotopes deuterium (D) and tritium (T).

The term “alkyl” as used herein refers to alkyl itself or a straight or branched chain radical of up to ten carbons. Useful alkyl groups include straight-chain or branched C₁-C₁₀ alkyl groups, preferably C₁-C₆ alkyl groups. In some embodiments, alkyl is C₁-C₄ alkyl. Typical C₁₀ alkyl groups include methyl, ethyl, propyl, isopropyl, butyl, sec-butyl, tent-butyl, 3-pentyl, hexyl and octyl groups, which may be optionally substituted.

The term “alkenyl” as used herein refers to a straight or branched chain radical of 2-10 carbon atoms, unless the chain length is limited thereto, wherein there is at least one double bond between two of the carbon atoms in the chain; preferably, C₂-C₆ alkenyl. Typical alkenyl groups include ethenyl, 1-propenyl, 2-propenyl, 2-methyl-1-propenyl, 1-butenyl and 2-butenyl.

The term “alkynyl” as used herein refers to a straight or branched chain radical of 2-10 carbon atoms, unless the chain length is limited thereto, wherein there is at least one triple bond between two of the carbon atoms in the chain; preferably, C₂-C₆ alkynyl. Typical alkynyl groups include ethynyl, 1-propynyl, 1-methyl-2-propynyl, 2-propynyl, 1-butynyl and 2-butynyl.

Useful alkoxy groups include oxygen substituted by the above mentioned C₁-C₁₀ alkyl groups, preferred C₁-C₆ alkyl groups or C₁-C₄ alkyl groups, e.g., methoxy, ethoxy, etc. The alkyl in the alkoxy groups may be optionally substituted. Substituents of alkoxy groups include, without limitation, halogen, morpholine, amino (including alkylamino and dialkylamino), and carboxy (including esters thereof).

Useful alkylthio groups include sulfur substituted by the above mentioned C₁-C₁₀ alkyl groups, preferred C₁-C₆ alkyl groups. The alkyl in the alkylthio groups may be optionally substituted. Also included are the sulfoxides and sulfones of such alkylthio groups.

Useful amino and optionally substituted amino groups include —NH₂, —NHR′ and —NR′R″, wherein R′ and R″ each are independently hydrogen, an optionally substituted C₁-C₁₀ alkyl, an optionally substituted cycloalkyl, an optionally substituted aryl or an optionally substituted heteroaryl; or R′ and R″ together with the N to which they are attached form an optionally substituted 4-7 membered cyclic amino group, which optionally comprises one or more (such as 2, 3) additional heteroatoms selected from O, N and S.

The term “aryl” as used herein by itself or as part of another group refers to monocyclic, bicyclic or tricyclic aromatic groups containing 6 to 14 carbon atoms. Aryl may be substituted by one or more substituents as described herein.

Useful aryl groups include C₆-C₁₄ aryl groups, preferably C₆-C₁₀ aryl groups. Typical C₆-C₁₄ aryl groups include phenyl, naphthyl, phenanthryl, anthracyl, indenyl, azulyl, biphenyl, biphenylene and fluorenyl.

The term “carbocycle (carbocyclic group)” as used herein include cycloalkyl and partially saturated carbocyclic groups. Useful cycloalkyl groups are C₃-C₈ cycloalkyl. Typical cycloallkyl groups include cyclopropyl, cyclobutyl, cyclopentyl, cyclohexyl and cycloheptyl. Carbocyclic group may be substituted by one or more substituents as described herein.

Useful partially saturated carbocyclic groups include cycloalkenyl groups, such as C₃-C₈ cycloalkenyl groups, e.g., cyclopentenyl, cycloheptenyl and cyclooctenyl.

Useful halo or halogen groups include fluoro, chloro, bromo and iodo.

Useful acylamino (acylamido) groups are any C₁-C₆ acyl (alkanoyl) attached to an amino nitrogen, e.g., acetamino, propionamido, butanoylamido, pentanoylamido and hexanoylamido, as well as aryl-substituted C₁-C₆ acylamino groups, e.g., benzoylamido. Useful acyl groups include C₁-C₆ acyl groups, such as acetyl. Acyl may be optionally substituted by group selected from aryl and halo, wherein the aryl may be optionally substituted. When acyl is substituted by halo, the number of halogen substituents may be in the range of 1-5. Examples of substituted acyls include chloroacetyl and pentafluorohenzoyl.

Useful acyloxy groups are any C₁-C₆ acyl (alkanoyl) attached to an oxygen (—O—), e.g., formyloxy, acetoxy, propionoyloxy, butanoyloxy, pentanoyloxy and hexanoyloxy.

The term “heterocyclyl (heterocyclic group)” as used herein refers to a saturated or partially saturated 3-7 membered rnonocyclic, 7-10 membered bicyclic;, tricyclic, or tetracyclic ring system having fused, bridging, and/or spiro 3-, 4-, 5-, 6-, 7-, or 8-membered rings, which consists of carbon atoms and one to four heteroatoms independently selected from O, N, and S, wherein the nitrogen and/or sulfur heteroatoms can be optionally oxidized and the nitrogen can be optionally quaternized, and the term also includes any bicyclic ring system in which any of the above-defined heterocyclic rings is fused to a benzene ring. The heterocyclic group can be substituted on carbon atom or nitrogen atom if the resulting compound is stable. Heterocyclic group may be substituted by one or more substituents as described herein. The heterocyclic groups mentioned herein also include 5 - to 8-member heterocyclic alkyl groups, that is, one or more ring C atoms in the cycloalkyl group are selected from N, O and S heterocyclic atom substitution.

Useful saturated or partially saturated heterocyclic groups include tetrahydrofuranyl, dihydrofuranyl, tetrahydrothienyl, dihydrothienyl, dihydroindolyl, tetrahydropyranyl, dihydropyranyl, piperidinyl, piperazinyl, oxazolidinyl, isoxazolidinyl, oxetanyl, azetidinyl, 1,4-diazepanyl, pyrrolinyl, pyrrolidinyl, imidazolidinyl, imidazolinyl, indolinyl, isoindolinyl, quinuclidinyl, morpholinyl, thiomorpholinyl, isochromanyl, chromanyl, pyrazolidinyl, pyrazolinyl, dithiazolyl, thiazolidinyl, isothiazolidinyl, thiazolidinyl, tetrahydroisoquinolinyl, tetrahydroisoquinolyl, tetronoyl, tetramoyl, and etc., which may be optionally substituted by one or more substituents as described herein.

The term “heteroaryl (heteroaromatic ring)” as used herein refers to a group having 5 to 14 ring atoms, with 6, 10 or 14 π electrons shared in a cyclic array. Ring atoms are carbon atoms and 1-3 heteroatoms selected from oxygen, nitrogen and sulfur. Heteroaryl may be optionally substituted by one or more substituents as described herein.

Useful heteroaryl groups include thienyl (thiophenyl), benzo[d]isothiazol-3-yl, benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, imidazolyl, pyrazolyl, pyridyl (pyridinyl, including without limitation 2-pyridyl, 3-pyridyl, and 4-pyridyl), pyrazinyl, pyrimidinyl, pyridazinyl, indolizinyl, isoindolyl, 3H-indolyl, indolyl, indazolyl, purinyl, isoquinolyl, quinolyl, phthalzinyl, naphthyridinyl, quinozalinyl, cinnolinyl, pteridinyl, carbazolyl, β-carbolinyl, phenanthridinyl, acridinyl, phenanthrolinyl, phenazinyl, isothiazolyl, phenothiazinyl, isoxazolyl, furazanyl, phenoxazinyl, 1,4-dihydroquinoxaline-2,3-dione, 7-amino-isocoumarin, pyrido[1,2-a]pyrimidin-4-one, tetrahydrocyclopenta[c]pyrazol-3-yl, benzoisoxazolyl such as 1,2-benzoisoxazol-3-yl, benzimidazolyl, 2-oxindolyl, thiadiazolyl, 2-oxobenzimidazolyl, imidazopyridazinyl, imidazopyridyl, triazolopyridazinyl, pyrazolopyrimidinyl, pyrrolopyrimidinyl, pyrrolopyridyl, pyrrolopyrazinyl or triazolopyrazinyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide,

In this disclosure, unless otherwise described, when substituted, the C₁-C₁₀ alkyl, cycloalkyl, heterocyclic alkyl, alkoxy, heterocyclic alkoxy, alkenyl, heterocyclic alkenyl, alkynyl, amino, acylamino, acyloxy, carboxyl, hydroxy, thiol, alkylthio, sulfonyl, sulfinyl, silyl, phosphocarboxyl, phosphoryl, carbocyclic group, heterocyclic group, aryl or heteroaryl as described in any embodiment herein may be substituted by one or more (such as 1, 2, 3, or 4) substituents selected from the group consisting of halogen, hydroxy, carboxyl, amino, nitro, cyano, C acylamino, C₁-C₆ acyloxy, C₁-C₆ alkoxy, aryloxy, alkylthio, C₁-C₆ alkyl, C₁-C₆ acyl, C₆-C₁₀ aryl, C₃-C₈ cycloalkyl, C₂-C₆ chain alkenyl, C₂-C₆ alkynyl, heterocyclic group, heteroaryl, methylenedioxy, ureido, thiol, azido, carbonyl, alkylsulfonyl, aminosulfonyl, dialkylaminosulfonyl, and alkyisulfiniyl, and the like. The substituent itself may also be optionally substituted. Preferred substituents include without limitation cyano, halo C₁-C₆ alkyl, halo, hydroxy, carboxyl, amino, C₁-C₆ acylamino, C₁-C₆ acyloxy, C₁-C₆ alkoxy, C₁-C₆ alkyl, C₁-C₆ acyl, and alkylsulfonyl.

It should be understood that in each embodiment, when the substituent is cyano, alkylsulfonyl, cycloalkyl, heterocyclic group, aryl or heteroaryl, the number of the substituent of cyano, alkylsulfonyl, heterocyclic, group, aryl or heteroaryl is usually 1.

Some of the compounds of the present disclosure may exist as stereoisomers including optical isomers. The disclosure includes all stereoisomers and the racemic mixtures of such stereoisomers as well as the individual enantiomers that may be separated according to methods that are well known to those of ordinary skill in the art.

Examples of pharmaceutically acceptable salts include inorganic and organic acid salts, such as hydrochloride, hydrobromide, phosphate, sulphate, citrate, lactate, tartrate, maleate, fumarate, mandelate and oxalate; and inorganic and organic base salts formed with bases, such as sodium hydroxy, tris(hydroxymethyl)aminomethane (TRIS, tromethamine) and N-methyl-glucamine.

Examples of prodrugs of the compounds of the disclosure include the simple esters of carboxylic acid-containing compounds (e.g., those obtained by condensation with a C₁-C₄ alcohol according to methods known in the art); esters of hydroxy containing compounds (e.g., those obtained by condensation with a C₁-C₄ carboxylic acid, C₃-C₆ diacid or anhydride thereof, such as succinic anhydride and fumaric anhydride according to methods known in the art); imines of amino containing compounds (e.g., those obtained by condensation with a C₁-C₄ aldehyde or ketone according to methods known in the art); carbamate of amino containing compounds, such as those described by Leu, et at., (J. Med. Chem. 42:3623-3628 (1999)) and Greenwald, et at., (J. Med. Chem. 42:3657-3667 (1999)); and acetals and ketals of alcohol-containing. compounds (e.g., those obtained by condensation with chloromethyl methyl ether or chloromethyl ethyl ether according to methods known in the art).

The compounds of this disclosure may be prepared using methods known to those skilled in the art, or the novel methods of this disclosure. Specifically, the compounds of this disclosure with Formula I (including Formulae II, III and IV) can be prepared as illustrated by the exemplary reaction in Scheme 1-2. Suzuki coupling of 4-bromo-1,2-dihydropyridazine-3,6-dione and boronic acid compounds under the catalysis of Pd(dppf)Cl₂ produced compound 2. Reaction of compound 2 in POCl₃ produced compound 3. Reaction of compound 3 and (R)-3-methylmorpholine in DIEA produced compound 4. Compound 6 can be prepared from compound 4 using the following two reaction schemes. (1) Scheme 1: reaction of compound 4 and Zn(CN)₂ under the catalysis of Pd₂(dba)₃ and DPPF in DMF produced compound 5-1. Reaction of compound 5-1 and CH₃MgI in THE under the protection of nitrogen at room temperature produced compound 6. (2) Scheme 2: reaction of compound 4 and tributyl(1-ethoxyvinyl)tin under the catalysis of Pd(PPh₃)₄, produced compound 5-2. Under the catalysis of TsOH, the ethyl group was removed from compound 5-2 to produce compound 6. Reaction of compound 6 and hydroxylamine hydrochloride in MeOH produced compound 7. Under the catalysis of Raney nickel, compound 7 was reduced to produce compound 8. Condensation of compound 8 with optionally substituted 1H-pyrazole-5-carboxylic acid under the catalysis of EDCI and HOBT produced compound 9. Reaction of compound 9 in POCl₃ produced the target compound. Wherein, R₂ in exemplary boronic acid compounds includes:

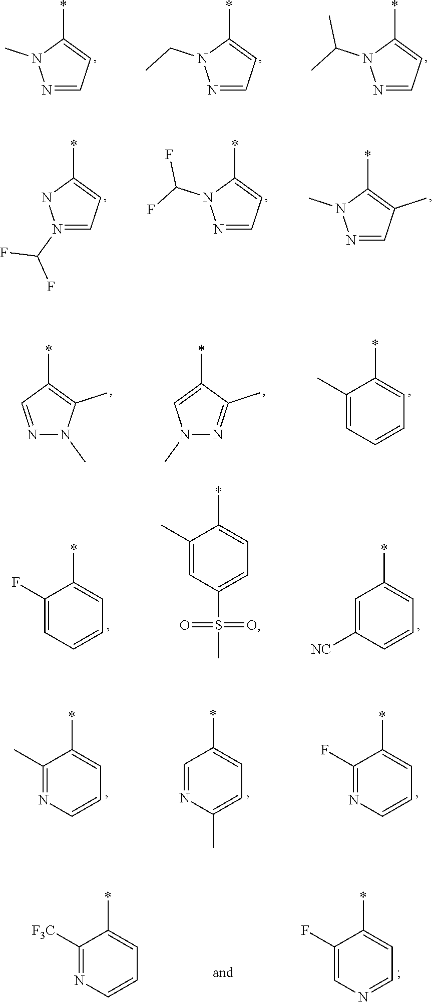

Exemplary R₂₂ is H or methyl, * refer to an attachment position of the group to the rest of the compound.

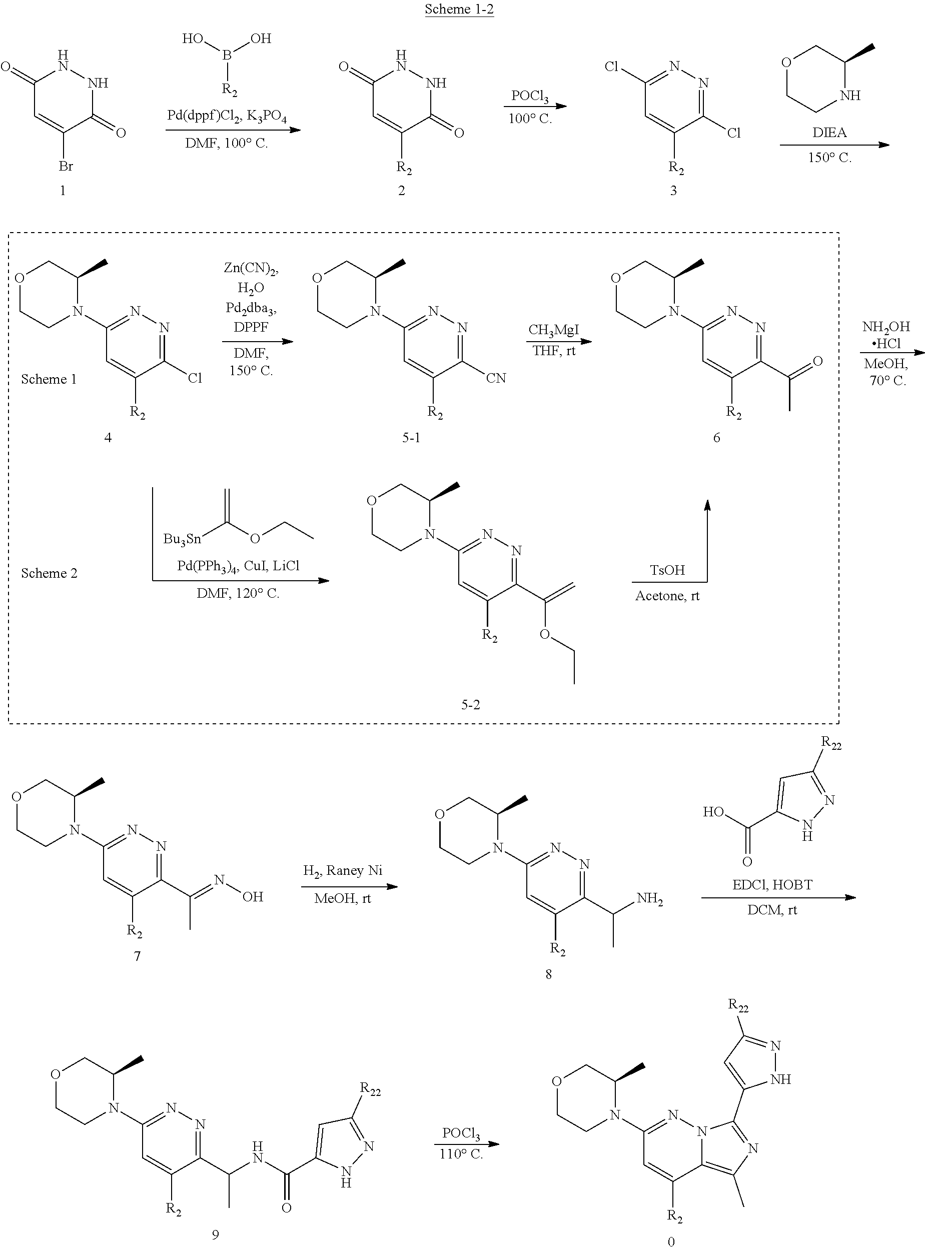

The compounds of this disclosure can be prepared as illustrated by the exemplary reaction in Scheme 3. Suzuki coupling of 4-bromo-1,2-dihydromidazine-3,6-dione and 2-(3,6-dihydro-2H-pyran-4-yl)-4,4,5,5-tetramethyl-1,3,2-dioxaborolane under the catalysis of Pd(dppf)Cl₂ produced compound 10. Under the catalysis of Pd/C, compound 10 was reduced to produce compound 11. Reaction of compound 11 in POCl₃ produced compound 12. Reaction of compound 12 and (R)-3-methylmorpholine in MBA produced compound 13. Under the catalysis of Pd₂(dba)₃ and DPPF, reaction of compound 13 and Zn(CN)₂ in DMF produced compound 14. Reaction of compound 14 and CH₃MgI in THF under the protection of nitrogen at room temperature produced compound 15. Reaction of compound 15 and hydroxylamine hydrochloride in MeOH produced compound 16. Under the catalysis of Raney nickel, compound 16 was reduced to produce compound 17. Condensation of compound 17 with 1H-pyrazole-5-carboxylic acid under the catalysis of EDCI and HOBT produced compound 18. Reaction of compound 18 in POCl₃ produced the target compound (R)-3-methyl-4-(5-methyl-7-(1H-pyrazol-5-yl)-4-(tetrahydro-2H-pyran-4-yl)imidazo[1,5-b]pyridazin-2-yl)morpholine.

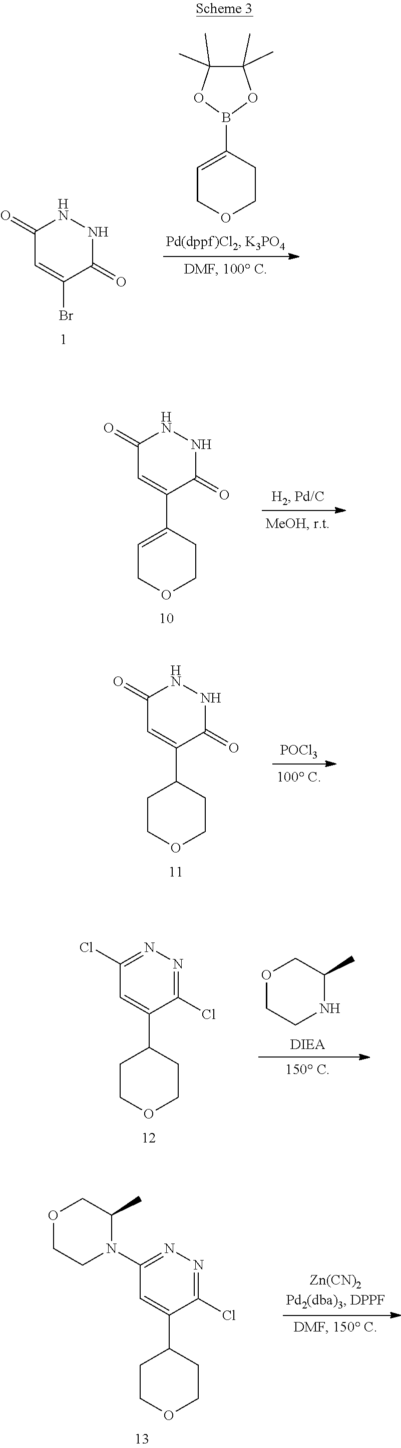

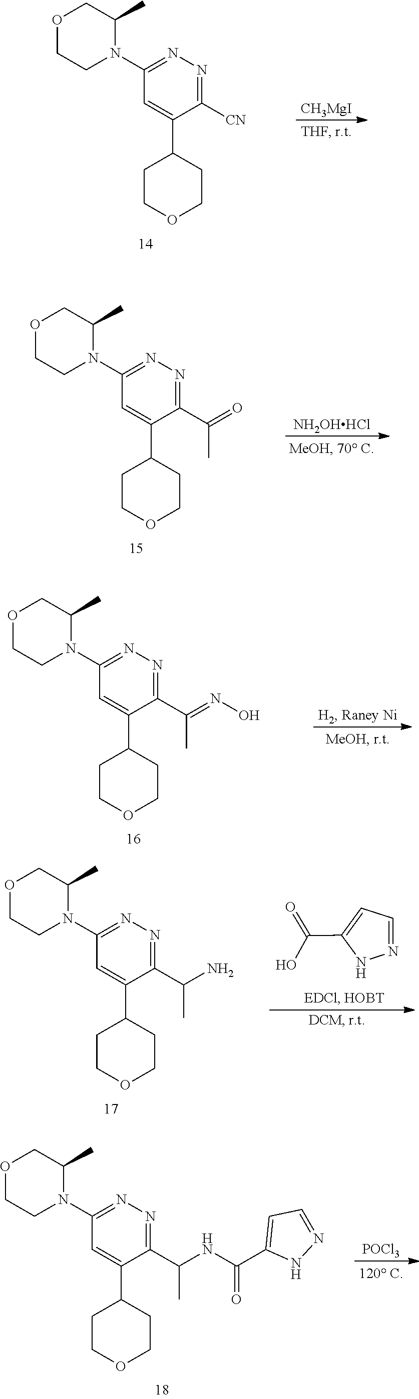

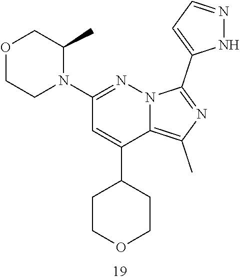 [SMILES: Cc1nc(-c2ccn[nH]2)n2nc(N3CCOC[C@H]3C)cc(C3CCOCC3)c12]

The compounds of this disclosure can be prepared as illustrated by the exemplary reaction in Scheme 4. (R)-3-Methyl-4-(4-(1-methyl-1H-pyrazol-5-yl)-7-(1H-pyrazol-5- yl)imidazo[1,5-b]pyridazin-2-yl)morpholine was brominated by NBS to produce (R)-4-(5-bromo-4-(1-methyl-1H-pyrazol-5-yl)-7-(1H-pyrazol-5-yl)imidazo[1,5-b]pyridazin-2-yl)-3-methylmorpholine. Coupling of (R)-4-(5-bromo-4-(1-methyl-1H-pyrazol-5-yl)-7-(1H-pyrazol-5-yl)imidazo[1,5-b]pyridazin-2-yl)-3-methylmorpholine and tributyl(vinyl)tin under the catalysis of Pd(PPh₃)₄ produced (R)-3-methyl-4-(4-(1-methyl-1H-pyrazol-5-yl)-7-(1H-pyrazol-5-yl)-5-vinylimidazo[1,5-b]pyridazin-2-yl)morpholine. Under the catalysis of Pd/C, (R)-3-methyl-4-(4-(1-methyl-1H-pyrazol-5-yl)-7-(1H-pyrazol-5-yl)-5-vinylimidazo[1,5-b]pyridazin-2-yl)morpholine was reduced to produce (R)-4-(5-ethyl-4-(1-methyl-1H-pyrazol-5-yl)-7-(1H-pyrazol-5-yl)imidazo[1,5-b]pyridazin-2-yl)-3-methylmorpholine.

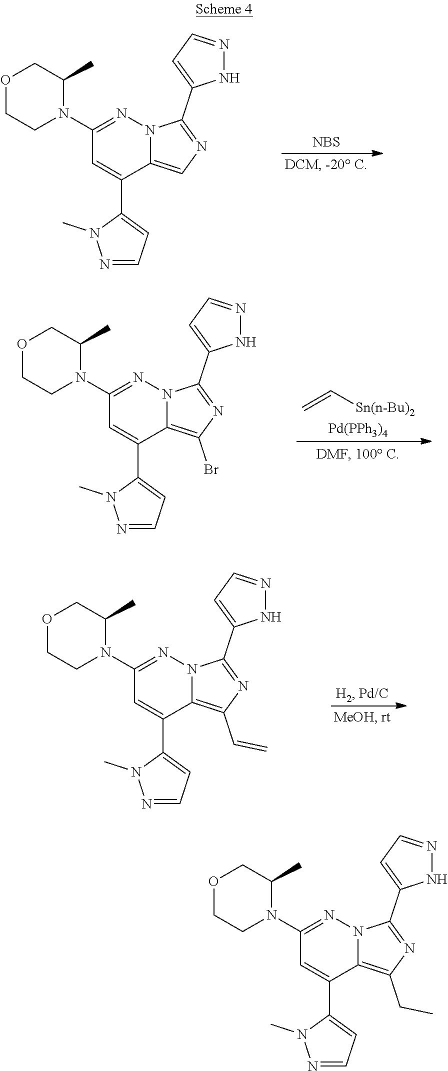

Other related compounds can be prepared using similar methods. For example, replacement of tributyl(vinyl)tin with isopropenyltributylstannane produced the target compound (R)-3-methyl-4-(4-(1-methyl-1H-pyrazol-5-yl)-5-(prop-1-en-2-yl)-7-(1H-pyrazol-5-yl)imidazo[1,5-b]pyridazin-2-yl)morpholine, and then the target compound (R)-4-(5-isopropyl-4-(1-methyl-1H-pyrazol-5-yl)-7-(1H-pyrazol-5-yl)imidazo[1,5-b]pyridazin-2-yl)-3-methylmorpholine can be prepared. Replacement of (R)-3 -Methyl -4-(4-(1-methyl-1H-pyrazol-5-yl)-7-(1H-pyrazol-5-yl)imidazo[1,5-b]pyridazin-2-yl)morpholine with (R)-3-methyl-4-(4-(1-methyl-1H-pyrazol-5-yl)-7-(1H-pyrazol-5-yl)imidazo[5,1f][1,2,4]triazin-2-yl)morpholine produced the target compound (R)-4-(5-bromo-4-(1-methyl-1H-pyrazol-5-yl)-7-(1H-pyrazol-5-yl)imidazo[5,1-f][1,2,4]triazin-2-yl)-3-methylmorpholine, and then the target compound (R)-3-methyl-4-(5-methyl-4-(1-methyl-1H-pyrazol-5-yl)-7-(1H-pyrazol-5-yl)imidazo[5,1-f][1,2,4]triazin-2-yl)morpholine can be prepared.

One important aspect of the present disclosure is the finding that the compounds of Formula I (including the compounds of Formulae II, III and IV as described herein) are kinase inhibitors, especially ATR kinase inhibitors. Therefore, the compounds of Formula I (including the compounds of Formulae II, III and IV as described herein) can be used to treat an ATR kinase-mediated related disease, such as cancer; or be used to prepare medicaments for the treatment of an ATR kinase-mediated related disease, such as cancer. In addition, an important and unexpected discovery is that when R₁ is halogen, an optionally substituted C₁-C₆ alkyl, an optionally substituted. C₃-C₆ cycloalkyl or an optionally substituted C₂-C₆ alkenyl, the compounds of Formula I (such as the compounds of Formula II, III or IV) are highly potent ATR kinase inhibitor with excellent oral absorption. Therefore, in some preferred embodiments, the present disclosure particularly relates to compounds in which R₁ is halogen, C₁-C₄ alkyl or C₂-C₄ alkenyl, more preferably compounds in which R₁ is halogen, C₁-C₃ alkyl or C₂-C₃ alkenyl, and use thereof to treat or prevent various clinical conditions caused by DDR function defects, or treat or prevent related diseases mediated by ATR kinase.

The present disclosure also includes methods for the treatment or prevention of kinase-mediated diseases, especially ATR kinase-mediated related diseases, comprising administering to an object (especially mammal, more specifically human) in need an effective amount of the compound of Formula I (including the compound of Formulae II, III and IV as described herein) or stereoisomers, tautomers, N-oxides, hydrates, solvates, isotope-substituted derivatives, or pharmaceutically acceptable salts thereof, or mixtures thereof, or prodrugs thereof, or a pharmaceutical composition comprising an effective amount of the compound of Formula I (including the compound of Formulae II, III and IV as described herein) or stereoisomers, tautomers, N-oxides, hydrates, solvates, isotope-substituted derivatives, or pharmaceutically acceptable salts thereof, or mixtures thereof, or prodrugs thereof.

In the disclosure, the kinase-mediated diseases include cancer, especially ATR kinase-mediated cancer. Preferably, the said ATR kinase-mediated cancer is deficient in DDR function. The ATR kinase-mediated diseases that can be treated or prevented by the methods or pharmaceutical compositions of the disclosure include without limitation liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, Wilms tumor, cervical cancer, testicular cancer, soft tissue sarcoma, primary macroglobulinemia, bladder cancer, chronic myeloid leukemia, primary brain cancer, malignant melanoma, non-small cell lung cancer, small cell lung cancer, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostate cancer.

Diseases of the present disclosure also includes those caused by excessive or abnormal cell proliferation, including proliferative or hyperproliferative diseases, such as myeloproliferative diseases, especially proliferative or hyperproliferative diseases caused by excessive or abnormal cell proliferation mediated by ATR kinase. Therefore, the disclosure also includes use of the compound of Formula I (including the compound of Formulae II, III and IV as described herein) or stereoisomers, tautomers, N-oxides, hydrates, solvates, isotope-substituted derivatives, or pharmaceutically acceptable salts thereof, or mixtures thereof, or prodrugs thereof, for the treatment or prevention of other diseases caused by excessive or abnormal cell proliferation, especially proliferative or hyperproliferative diseases caused by excessive or abnormal cell proliferation mediated by ATR kinase.

In practicing the therapeutic methods, effective amounts of pharmaceutical preparations are administered to an individual exhibiting the symptoms of one or more of these disorders. The pharmaceutic preparations comprise therapeutically effective concentrations of the compounds of Formula I, II, III, or IV, or stereoisomers, tautomers, N-oxides, hydrates, solvates, isotope-substituted derivatives, or pharmaceutically acceptable salts thereof, or mixtures thereof, or prodrugs thereof, formulated for oral, intravenous, local or topical application, for the treatment of cancer and other diseases. The amounts are effective to ameliorate or eliminate one or more symptoms of the disorders, An effective amount of a compound for treating a particular disease is an amount that is sufficient to ameliorate or in some manner reduce the symptoms associated with the disease. Such amount may be administered as a single dosage or may be administered according to an effective regimen. The amount may cure the disease but, typically, is administered in order to ameliorate the symptoms of the disease. Typically, repeated administration is required to achieve the desired amelioration of symptom.

In another embodiment, there is provided a pharmaceutical composition comprising a compound of Formula I, II, III, or IV, or stereoisomers, tautomers, N-oxides, hydrates, solvates, isotope-substituted derivatives, or pharmaceutically acceptable salts thereof, or mixtures thereof, or prodrugs thereof, as an ATR kinase inhibitor, and a pharmaceutically acceptable carrier.

Another embodiment of the present disclosure is directed to a pharmaceutical composition effective to treat cancer comprising a compound of Formula I, II, III, or IV, or stereoisomers, tautomers, N-oxides, hydrates, solvates, isotope-substituted derivatives, or pharmaceutically acceptable salts thereof, or mixtures thereof, or prodrugs thereof, which functions as a kinase inhibitor, in combination with at least one known anticancer agent or a pharmaceutically acceptable salt thereof. In particular, the compound herein can be combined with other anticancer drugs related to the mechanism of DNA damage and repair, including PARP inhibitors, such as olaparib, niraprib, rucaparib, talazoparib, pamiparib, fluzoparib and senaparib; TNKS inhibitors; HDAC inhibitors such as Vorinostat, Romidepsin, Panobinostat and Belinostat; and so on, And the compound herein can be combined with other anticancer drugs related to cell division detection sites, including CHK1/2 inhibitors, CDK4/6 inhibitors such as Palbociclib ATM inhibitors, Wee1 inhibitors, MYT1 inhibitors, DNA-PK inhibitors, and so on. And combination with other targeted anti-cancer drugs, including USP1 inhibitors, PRMT5 inhibitors, Pole inhibitors, RAD51 inhibitors, and so on. Other known anticancer agents which may be used for anticancer combination therapy include, but are not limited to alkylating agents, such as busulfan, melphalan, chlorambucil, cyclophosph amide, ifosfamide, temozolomide, bendamustine, cis-platin, mitomycin C, bleomycin and carboplatin; topoisomerase I inhibitors, such as camptothecin, irinotecan and topotecan; topoisomerase II inhibitors, such as doxorubicin, epirubicin, aclacinomycin, mitoxantrone, elliptinium and etoposide; RNA/DNA antimetabolites, such as 5-azacytidine, gemcitabine, 5-fluorouracil, capecitabine and methotrexate; DNA antimetabolites, such as 5-fluoro-2′-deoxy-uridine, fludarabine, nelarabine, ara-C, pralatrexate, pemetrexed, hydroxyurea and thioguanine; antimitotic agent such as colchicine, vinblastine, vincristine, vinorelbine, paclitaxel, ixabepilone, cabazitaxel and docetaxel; antibodies such as McAb, panitumumab, necitumumab, nivolumab, pembrolizumab, ramucirumab, bevacizumab, pertuzualab, trastuzumab, cetuximab, obinutuzumab, ofatumumab, rituximab, alemtuzumab, ibritumomab, tositumomab, brentuximab, daratumumab, elotuzumab, T-DMI, Ofatumumab, Dinutuximab, Blinatumomab, ipilimumab, avastin, herceptin and mabthera; kinase inhibitors such as imatinib, gefitinib, erlotinib, osimertinib, afatinib, ceritinib, alectinib, crizotinib, erlotinib, lapatinib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, Idelalisib, pazopanib, Torisel and everolimus. Other known anticancer agents which may be used for anticancer combination therapy include tamoxifen, letrozole, fulvestrant, mitoguazone, octreotide, retinoic acid, arsenic, zoledronic acid, bortezomib, carfilzomib, Ixazomib, vismodegib, sonidegib, denosumab, thalidomide, lenalidomide, Venetociax, Aldesleukin (recombinant human interleukin-2) and Sipueucel-T (prostate cancer treatment vaccine).

In practicing the methods of the present disclosure, the compound of the disclosure may be administered together with at least one known anticancer agent in a unitary pharmaceutical composition. Alternatively, the compound of the disclosure may be administered separately from at least one known anticancer agent. In one embodiment, the compound of the disclosure and at least one known anticancer agent are administered substantially simultaneously, i.e. all agents are administered at the same time or one after another, provided that compounds reach therapeutic levels in the blood at the same time. In another embodiment, the compound of the disclosure and at least one known anticancer agent are administered according to individual dose schedule, provided that the compounds reach therapeutic levels in the blood.

Another embodiment of the present disclosure is directed to a bioconjugate, which functions as a kinase inhibitor, that comprises a compound described herein and is effective to inhibit tumor. The bioconjugate that inhibits tumor is consisted of the compound described herein and at least one known therapeutically useful antibody, such as trastuzumab or rituximab, or growth factor, such as EGF or FGF, or cytokine, such as IL-2 or IL-4, or any molecule that can bind to cell surface. The antibodies and other molecules could deliver the compound described herein to its targets, making it an effective anticancer agent. The bioconjugates could also enhance the anticancer effect of the therapeutically useful antibodies, such as trastuzumab or rituximab.

Another embodiment of the present disclosure is directed to a pharmaceutical composition effective to inhibit tumor comprising the kinase inhibitor of Formula I (including the compound of Formulae II, III and IV as described herein), or stereoisomers, tautomers, N-oxides, hydrates, solvates, isotope-substituted derivatives, or pharmaceutically acceptable salts thereof, or mixtures thereof, or prodrugs thereof, in combination with radiation therapy. In this embodiment, the compound of the disclosure may be administered at the same time as the radiation therapy or at a different time.

Yet another embodiment of the present disclosure is directed to a pharmaceutical composition effective for post-surgical treatment of cancer, comprising the kinase inhibitor of Formula I, I, III, or IV, or stereoisomers, tautomers, N-oxides, hydrates, solvates, isotope-substituted derivatives, or pharmaceutically acceptable salts thereof, or mixtures thereof, or prodrugs thereof. The disclosure also relates to a method of treating cancer by surgically removing tumor and then treating the mammal with the pharmaceutical composition described herein.

Pharmaceutical compositions of this disclosure include all pharmaceutical preparations which contain the compounds of the present disclosure in an amount that is effective to achieve its intended purpose. While individual needs vary, determination of optimal amounts of each component in the pharmaceutical preparations is within the skill of the art. Typically, the compounds or the pharmaceutically acceptable salt thereof may be administered to mammals, orally at a dose of about 0.0025 to 50 mg per kg body weight per day. Preferably, from approximately 0.01 mg/kg to approximately 10 mg/kg body weight is orally administered. If a known anticancer agent is also administered, it is administered in an amount that is effective to achieve its intended purpose. The optimal amounts of such known anticancer agents are well known to those skilled in the art.

The unit oral dose may comprise from approximately 0.01 to approximately SO mg, preferably approximately 0.1 to approximately 10 mg of the compound of the disclosure. The unit dose may be administered one or more times, with one or more tablets daily, each containing from approximately 0.1 to approximately 50 mg, conveniently approximately 0.25 to 10 mg of the compound of the disclosure or its solvates.

In a topical formulation, the compound of the disclosure may be present at a concentration of approximately 0.01 to 100 mg per gram of carrier.

The compound of the disclosure may be administered as a raw chemical. The compounds of the disclosure may also be administered as part of a suitable pharmaceutical preparation containing pharmaceutically acceptable carriers (comprising excipients and auxiliaries), which facilitate the processing of the compounds into pharmaceutically acceptable preparations. Preferably, the pharmaceutical preparations, particularly oral preparations and those used for the preferred administration, such as tablets, dragees, and capsules, as well as solutions suitable for injection or oral administration, contain from approximately 0.01% to 99%, preferably from approximately 0.25% to 75% of active compound(s), together with excipient(s).

Also included within the scope of the present disclosure are the non-toxic pharmaceutically acceptable salts of the compounds of the present disclosure. Acid addition salts are formed by mixing a solution of the compounds of the present disclosure with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Base addition salts are formed by mixing a solution of the compounds of the present disclosure with a solution of a pharmaceutically acceptable non-toxic base, such as sodium hydroxide, potassium hydroxide, choline hydroxide, sodium carbonate, tris(hydroxymethyl)aminomethane, N-methyl-glucamine and the like.

The pharmaceutical preparations of the disclosure may be administered to any mammal, so long as they may experience the therapeutic effects of the compounds of the disclosure. Foremost among such mammals are humans and veterinary animals, although the disclosure is not intended to be so limited.

The pharmaceutical preparations of the present disclosure may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively or concurrently, administration may be by oral route. The dosage administered will be dependent upon the age, health, and weight of the recipient, type of concurrent treatment, frequency of treatment, and the nature of the effect desired.

The pharmaceutical preparations of the present disclosure are manufactured in a known manner, e.g., by means of conventional mixing, granulating, dragee-making, dissolving, or lyophilizing processes. Pharmaceutical preparations for oral use may be obtained by combining the active compounds with solid excipients, optionally grinding the resulting mixture, processing the mixture of granules after adding suitable auxiliaries if desired or necessary, thereby obtaining tablets or dragee cores.

Suitable excipients are, in particular, fillers, such as saccharides, e.g. lactose or sucrose, mannitol or sorbitol cellulose preparations and/or calcium phosphates, e.g. tricalcium phosphate or calcium hydrogen phosphate; as well as binders, such as starch paste, including, e.g., maize starch, wheat starch, rice starch, potato starch, gelatin, tragacanth, methylcellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, in particular, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropylmethylcellulose phthalate, are used. Dyes or pigments may be added to the tablets or dragee coatings, e.g., for identification or in order to characterize combinations of active compound doses.

Other pharmaceutical preparations, which may be used orally, include push-fit capsules made of gelatin, as well as soft, sealed capsules made of gelatin and a plasticizer, such as glycerol or sorbitol. The push-fit capsules may contain the active compounds in the form of granules, which may be mixed with fillers, such as lactose; binders, such as starches; and/or lubricants, such as talc or magnesium stearate and stabilizers. In soft capsules, the active compounds are preferably dissolved or suspended in suitable liquids, such as fatty oils, or liquid paraffin. In addition, stabilizers may be added.

Suitable formulations for parenteral administration include aqueous solutions of the active compounds, e.g., aqueous solutions and alkaline solutions of water-soluble salts. In addition, suspensions of the active compounds as appropriate oily injection suspensions may be administered. Suitable lipophilic solvents or vehicles include fatty oils, e.g., sesame oil, or synthetic fatty acid esters, e.g., ethyl oleate or triglycerides or polyethylene glycol-400, or cremophor, or cyclodextrins. Aqueous injection suspensions may contain substances which increase the viscosity of the suspension, e.g., sodium carboxymethyl cellulose, sorbitol, and/or dextran. Optionally, suspension stabilizers may also be contained.

In accordance with one aspect of the present disclosure, compounds of the disclosure are employed in topical and parenteral formulations and are used for the treatment of skin cancer.

The topical formulations of this disclosure are formulated preferably as oils, creams, lotions, ointments and the like by choice of appropriate carriers. Suitable carriers include vegetable or mineral oils, white petrolatum (white soft, paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The present disclosure also involves use of the compounds of the disclosure for the preparation of medicaments for the treatment of clinical symptoms in response to the effect of inhibiting the activity of kinases (especially ATR kinase). These medicaments may include the above-mentioned pharmaceutical compositions.

The following examples are illustrative, but not limiting, of the method and compositions of the present disclosure. Other suitable modifications and adaptations of the variety of conditions and parameters normally encountered in clinical therapy and which are Obvious to those skilled in the art are within the spirit and scope of the disclosure.

### EXAMPLES

### General Remarks

All reagents were of commercial quality. Solvents were dried and purified by standard methods. Mass spectrum analyses were recorded on a Platform II (Agilent 6110) quadrupole mass spectrometer fitted with an electrospray rinterface. ¹H NMR spectra was recorded at 400 MHz, on a Brucker Ascend 400 apparatus. Chemical shifts were recorded in parts per million (ppm) downfield from TMS (0.00 ppm), and J coupling constants were reported in hertz (Hz).

### Example 1

### (R)-3-methyl-4-(5methyl-4-(1-methyl-1H-pyrazol-5-yl)-7-(1H-pyrazol-5-yl)imidazo[1,5-b]pyridazin-2-yl)morpholine

a) Preparation of 4-(1-methyl-1H-pyrazol-5-yl)-1,2-dihydropyridazine-3,6-dione: To a solution of 4-bromo-1,2-dihydropyridazine-3,6-dione (70.0 g, 366.3 mmol), (1-methyl-1H-pyrazol-5-yl)boronic acid (91.6 g, 732.6 mmol) and 1 M aq. K₃PO₄ (1000 mL) in DMF (1000 mL) was added Pd(dppf)Cl₂ (8.0 g, 11.0 mmol). The system was evacuated and backfilled with N₂ three times and stirred at 100° C. for 16 h. After completion, the mixture was cooled down to room temperature and the aqueous phase was decanted. The organic phase was filtered and the filtrate was evaporated to give the crude product which was washed with DCM (500 mL×3). The solid collected was dried to give the crude target compound (200.0 g, crude, yellow solid) which was used for the next step directly. LC-MS(ESI): 193.30 [M+H]⁺. ¹H NMR (400 MHz, D₂O): δ 7.37 (brs, 1H), 6.80 (brs, 1H), 6.26 (brs, 1H), 3.56 (s, 3H).

b) Preparation of 3,6-dichloro-4-(1-methyl-1H-pyrazol-5-yl)pyridazine: A solution of 4-(1-methyl-1H-pyrazol-5-yl)-1,2-dihydropyridazine-3,6-dione (80.0 g, 0.4 mol, crude) in POCl₃ (400 mL) was stirred at 100° C. for 16 h. After completion, the solvent was removed and the residue was quenched with water (200 mL) slowly, and the pH of the resulting mixture was adjusted to 9 with aq. ammonium hydroxide (˜25% w/w). The mixture was extracted with EA (300 mL×3). The organic phase was washed with brine, dried over anhydrous sodium sulfate and evaporated to give the crude product (27.0 g, yield: 81%, 2 steps, black solid) which was used for the next step directly. LC-MS(ESI): 229.20 [M+H]⁺. ¹H NMR (400 MHz CDCl₃): δ 7.61 (d, J=1.3 Hz, 1H), 7.51 (s, 1H), 6.49 (d, J=1.3 Hz, 1H) 3.83 (s, 3H).

c) Preparation of (R)-4-(6-chloro-5-(1-methyl-1H-pyrazol-5-yl)pyridazin-3-yl)-3-methylmorpholine: To a solution of 3,6-dichloro-4-(1-methyl-1H-pyrazol-5-yl)pyridazine (50.0 g, 219.0 mmol) in MBA (84.7 g, 657.0 mmol) was added (R)-3-methylmorpholine (26.5 g, 262.8 mmol). The mixture was stirred at 150° C. for 6 h. After completion of the reaction, to the mixture was added water (1000 mL) and the mixture was extracted with EA (400 mL×3). The combined organic phase was washed with brine, dried over anhydrous sodium sulfate and concentrated under reduced pressure to afford the crude product (59.0 g, yield: 92%, black solid) which was used for the next step directly. LC-MS(ESI): 294.30 [M+H]³⁰ . ¹H NMR (400 MHz, CDCl₃): δ 7.58-7.53 (m, 1H), 6.78 (s, 1H), 6.36 (s, 1H), 4.31 (q, J=5.5, 4.3 Hz, 1H), 4.10-4.00 (m, 1H), 3.96 (d, J=13.1 Hz, 1H), 3.83-3.77 (m, 5H), 3.64 (td, J=12.0, 2.9 Hz, 1H), 3.35 (td, J=13.0, 12.6, 3.4 Hz, 1H), 1.32 (d, J=6.7 Hz, 3H).

d) Preparation of (R)-4-(1-methyl-1H-pyrazol-5-yl)-6-(3-methylmorpholino)pyridazine-3-carbonitrile: Under the protection of nitrogen, to a solution of (R)-4-(6-chloro-5-(1-methyl-1H-pyrazol-5-yl)pyridazin-3-yl)-3-methylmorpholine (0.5 g, 1.7 mmol) in DMF (10 mL) was added Zn(CN)₂ (0.4 g 3.4 mmol), 1,1′-Bis(diphenylphosphino)ferrocene (DPPF, 0.2 g, 0.36 mmol), Pd₂(dba)₃ (160 mg, 0.17 mmol) and water. The mixture was stirred at 150° C. for 6 h. After the solvent was removed, the residue was purified by chromatography over silica gel (EtOAc/PE, 10 to 40%) to afford the product (0.4 g, yield: 82%, slightly red oil). LC-MS(ESI): 285.30 [M+H]⁺. ¹H NMR (400 MHz, CDCl₃): δ 7.59 (d, J=1.8 Hz, 1H), 6.66 (s, 1H), 6.53 (d, J=1.8 Hz, 1H), 4.54-4.43 (m, 1H), 4.23-4.13 (m, 1H), 4.08 (dd, J=14.8, 5.7 Hz, 1H), 3.89 (s, 3H), 3.84 (brs, 1H), 3.78 (dd, J=12.0, 2.8 Hz, 1H), 3.64 (td, J=12.4, 2.8 Hz, 1H), 3.49-3.38 (m, 1H), 1.38 (d, J=6.8 Hz, 3H).

e) Preparation of (R)-1-(4-(1-methyl-1H-pyrazol-5-yl-6-(3-methylmorpholino)pyridazin-3-yl)ethanone: To a solution of (R)-4-(1-methyl-1H-pyrazol-5-yl)-6-(3-methylmorpholino)pyridazine-3-carbonitrile (3.3 g, 11.6 mmol) in THF (20 mL) was added CH₃MgI (3.0 M in Et₂O, 11.0 mL, 11.6 mmol) under N₂. The mixture was stirred at room temperature for 10 min. After completion, the mixture was quenched with aqueous saturated ammonium chloride (20 mL) and the mixture was extracted with EA (40 mL 3). The organic phase was separated and dried, concentrated to give the residue which was purified by chromatography over silica gel (petroleum ether: ethyl acetate=10:1˜1:1) to afford the product (1.3 g, yield: 37%, brown solid). LC-MS(ESI): 302.10 [M+H]⁺.

f) Preparation of 1-(4-(1-methyl-1H-pyrazol-5-yl)-6-(3-methylmorpholino)pyridazin-3-yl)ethanone oxime: To a solution of (R)-1-(4-(1-methyl-1H-pyrazol-5-yl)-6-(3-methylmorpholino)pyridazin-3-yl)ethanone (500.0 mg, 1.7 mmol) in MeOH (50 mL) was added hydroxylamine hydrochloride (1.2 g, 16.6 mmol) under the protection of N₂. The mixture was stirred at 70° C. overnight. After reaction completion, to the mixture was added water (50 mL) and the mixture was extracted with EA (50 mL×3). The combined organic phase was separated and dried, concentrated to afford the crude product (430.0 mg, yield: 82%, brown solid). LC-MS(ESI): 317.15 [M+H]⁺.

(g) Preparation of 1-(4-(1-methyl-1H-pyrazol-5-yl)-6-(®-3 methylmorpholi31therione31ezin-3-yl)ethanamine: To a solution of 1-(4-(1-methyl-1H-pyrazol-5-yl)-6-(3-methylmorpholi31thenone31ezin-3-yl)ethanone oxime (330.0 mg, 1.0 mmol) in MeOH (5 mL) was added Raney Ni (˜3.3 g). The mixture was stirred at room temperature overnight under H₂ atmosphere. After reaction completion, the mixture was filtered over celite and the solid was washed with MeOH (20 mL×5). The filtrate was combined and concentrated to afford the crude product (210.0 mg, yield: 67%, gray solid). LC-MS(ESI): 303.10 [M+H]⁺.

h) Preparation of N-(1-(4-(1-methyl-1H-pyrazol-5-yl)-6-((R)-3-methylmorpholino)pyridazin-3-yl)ethyl)-1H-pyrazole-5-carboxamide: To a solution of 1-(4-(1-methyl-1H-pyrazol-5-yl)-6-((R)-3-methylmorpholino)pyridazin-3-yl)ethanamine (210.0 mg, 0.7 mmol) in DCM (6 mL) was added 1H-pyrazole-5-carboxylic acid (93.0 mg, 0.8 mmol), EDCI (173.0 mg, 0.9 mmol) and HOBT (10.0 mg, 0.07 mmol). The mixture was stirred at room temperature overnight. After reaction completion, to the mixture was added water (10 mL) and the mixture was extracted with DCM (10 mL/3). The combined organic phase was washed with brine, dried over anhydrous sodium sulfate and concentrated. The residue was triturated with EA (3 mL) to give the target product (160.0 mg, yield: 58%, white solid). LC-MS(ESI): 397.20 [M+H]⁺.

i) Preparation of (R)-3-methyl-4-(5-methyl-4-(1-methyl-1H-pyrazol-5-yl)-7-(1H-pyrazol-5-yl)imidazo[1,5-b]pyridazin-2-yl)morpholine: A solution of N-(1-(4-(1-methyl-1H-pyrazol -5-yl)-6-((R)-3 -methylmorpholino)pyridazin-3-yl)ethyl)-1H-pyrazole-5-carboxamide (50.0 mg, 0.13 mmol) in POCl₃ (3 mL) was stirred at 110° C. for 4 h. After reaction completion, the solvent was removed. To the mixture was added water (5 mL) and the pH was adjusted to 8 with aq. ammonium hydroxide (25% w/w). The mixture was extracted with DCM (10 mL×2). The combined organic phase was washed with brine, dried over anhydrous sodium sulfate and concentrated. The residue was purified by Prep-HPLC (C18, CH₃CN/H₂O, 15-40%, 0.1% HCOOH) to afford the target compound (15.0 mg, yield: 32%, slightly yellow powder).

### Example 2

### (R)-4-(4-(1-ethyl-1H-pyrazol-5-yl)-5-methyl-7-(1H-pyrazol-5-yl)imidazo[1,5-b]pylidazin-2-yl)-3-methylmorpholine

a) Preparation of (R)-4-(6-(1-ethoxyvinyl)-5-(1-ethyl-1H-pyrazol-5-yl)pyridazin-3-yl)-3-methylmorpholine: To a solution of (R)-4-(6-chloro-5-(1-ethyl-1H-pyrazol-5-yl)pyridazin-3-yl)-3-methylmorpholine (Prepared by the similar method of Example 1a-1c, 100.0 mg, 0.3 mmol) in DMF (4 mL) was added CuI (6.0 mg, 11.0 mL, 0.03 mmol), Pd(PPh₃)₄ (33.0 mg, 0.3 mmol), LiCl (42.0 mg, 1.0 mmol) and tributyl(1-ethoxyvinyl)tin (590.0 mg, 1.6 mmol) under the protection of N₂. The mixture was stirred at 100 ° C. for 5 hours. After reaction completion, the mixture was quenched with aqueous saturated ammonium chloride (10 mL) and the mixture was extracted with EA (20 mL×3). The organic phase was separated and dried, concentrated to give the residue which was purified by chromatography over silica gel (petroleum ether: ethyl acetate=5:1˜1:1) to afford the target product (60.0 mg, yield: 54%, yellow solid). LC-MS(ESI): 344.15 [M+H]⁺.

b) Preparation of (R)-1-(4-(1-ethyl-1H-pyrazol-5-y)-6-(3-methylmorpholino)pyridazin-3-yl)ethan-1-one: To a solution of (1))-4-(6-(1-ethoxyvinyl)-5-(1-ethyl-1H-pyrazol-5-yl)pyridazin-3-yl)-3-methylmorpholine (300.0 mg, 0.9 mmol) in acetone (5 ml) was added TsOH (326.0 mg, 1.7 mmol). The mixture was stirred at room temperature for 30 min. After reaction completion, the solvent was concentrated to give the residue which was purified by chromatography over silica gel (petroleum ether: ethyl acetate=5:1-1:1) to afford the target product (270.0 mg, yield: 98%, yellow solid). LC-MS(ESI): 316.35 [M+H]⁺.

c) Preparation of (R)-1-(4-(1-ethyl-1H-pyrazol-5-yl)-6-(3-methylmorpholino)-pyridazin-3-yl)ethan-1-one oxime: To a solution of (R)-1-(4-(1-ethyl-1H-pyrazol-5-yl)-6-(3-methylmorpholino)pyridazin-3-yl)ethan-1-one (580.0 mg, 1.8 mmol) MeOH (10 mL) was added hydroxylamine hydrochloride (1.3 g, 18.6 mmol) under the protection of N₂. The mixture was stirred at 70° C. overnight. After reaction completion, the mixture was evaporated and the residue was dissolved in water (10 mL) and EA (20 mL). The organic phase was separated and dried, concentrated to afford the crude target product (150.0 mg, yield: 25%, brown solid). LC-MS(ESI): 331.25 [M+H]⁺.

d) Preparation of 1-(4-(1-ethyl-1H-pyrazol-5-yl)-6-((R)-3-methymorpholino)pyridazin-3-yl)ethan-1-amine: To a solution of (R)-1-(4-(1-ethyl-1H-pyrazol-5-yl)-6-(3-methylmorpholino)pyridazin-3-yl)ethan-1-one oxime (150.0 mg, 0.5 mmol) in MeOH (5 mL) was added Raney Ni (˜0.8 g, w/w). The system was evacuated and backfilled with H₂ three times and stirred at rt for 16 h under H₂ atmosphere. After reaction completion, the mixture was filtered over Celite and the cake was washed with MeOH (10 mL×3). The filtrate was combined and concentrated to afford the crude target product (120 mg, black solid). LC-MS(ESI): 317.30 [M+H]⁺.

e) Preparation of N-(1-(4-(1-ethyl-1H-pyrazol-5-yl)-6-((R)-3-methylmorpholino)pyridazin-3-yl)ethyl)-1H-pyrazole-5-carboxamide: To a solution of 1-(4-(1-ethyl-1H-pyrazol-5-yl)-6((R)-3-methylmorpholino)pyridazin-3-yl)ethan-1-amine (120.0 mg crude, 0.4 mmol) in DCM (4 mL) was added 1H-pyrazole-5-carboxylic acid (51.0 mg, 0.5 mmol), EDCI (95.0 mg, 0.5 mmol) and HOBT (5.0 mg, 0.04 mmol). The mixture was stirred at room temperature overnight. After reaction completion, DCM (10 mL) was added and the mixture was washed with water (10 mL×2). The DCM layer was dried over anhydrous sodium sulfate and concentrated. The residue was triturated with EA (4 mL) to give the target product (70.0 mg, yield: 38%, 2 steps, white solid). LC-MS(ESI): 411.25[M+H]⁺.

Preparation of (R)-4-(4-(1-ethyl-1H-pyrazol-5-yl)-5-methyl-7-(1H-pyrazol-5-yl)imidazo[1,5-b]pyridazin-2-yl)-3-methylmorpholine: A solution of N-(1-(4-(1-ethyl-1H-pyrazol-5-yl)-6-((R)-3-methylmorpholino)pyridazin-3-yl)ethyl)-1H-pyrazole-5-carboxamide (70.0 mg, 17.0 mmol) in POCl₃ (7 mL) was stirred at 120° C. for 4 h. After reaction completion, the solvent was removed under reduced pressure. To the mixture was added water (10 mL) and the pH was adjusted to 8 with aq. ammonium hydroxide (25% w/w). The mixture was extracted with DCM (10 mL×2). The combined organic phase was washed with brine, dried over anhydrous sodium sulfate and concentrated. The crude product was purified by Prep-TLC (PCM:MeOH=10:1) to afford the target compound (26.0 mg, yield: 39%, yellow solid).

The following compounds of Examples 3-6 were prepared using the synthesis method similar to that described in Example 1 or Example 2.

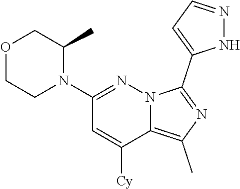

| | | | LC-MS | |
|---|---|---|---|---|
| Example | Cy | MW | (ESI) | ¹H NMR (400 MHz) |
| 1 | 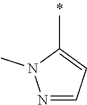 | 378.44 | 379.15 [M + H]⁺ | DMSO-d₆: δ 7.69 (s, 1H), 7.61 (s, 1H), 7.10 (s, 1H), 6.76 (s, 1H), 6.55 (s, 1H), 4.33 (d, J = 7.6 Hz, 1H), 3.98 (d, J = 11.4 Hz, 1H), 3.88 (d, J = 13.3 Hz, 1H), 3.72 (d, J = 11.9 Hz, 7H), 1.92 (s, 3H), 1.23 (d, J = 6.6 Hz, 3H). |
| 2 | 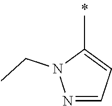 | 392.47 | 393.35 [M + H]⁺ | CDCl₃: δ 7.73 (s, 1H), 7.64 (s, 1H), 7.10 (s, 1H), 6.40 (s, 1H), 6.33 (s, 1H), 4.13-4.05 (m, 4H), 3.89- 3.75 (m, 4H), 3.69 (t, J = 12.0 Hz, 1H), 3.46 (t, J = 12.9 Hz, 1H), 2,03 (s, 3H), 1.41-1.33 (m, 6H). |
| 3 | 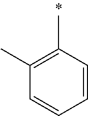 | 388.48 | 389.15 [M + H]⁺ | CDCl₃: δ 7.71 (s, 1H), 7.40 (t, J = 7.5 Hz, 1H), 7.31 (t, J = 8.3 Hz, 1H), 7.27-7.17 (m, 2H), 7.07 (s, 1H), 6.23 (s, 1H), 4.19-3.98 (m, 2H), 3.91-3.75 (m, 3H), 3.69 (t, J = 12.2 Hz, 1H), 3.45 (t, J = 12.4 Hz, 1H), 2.18 (s, 3H), 1.88 (s, 3H), 1.37 (s, 3H). |
| 4 | 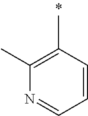 | 389.46 | 390.30 [M + H]⁺ 388.20 [M − H]⁻ | CD₃OD: δ 8.57 (s, 1H), 7.80-7.75 (m, 2H), 7.44 (s, 1H), 7.18 (s, 1H), 6.63 (s, 1H), 4.31 (s, 1H), 4.03 (d, J = 11.6 Hz, 1H), 3.89 (t, J = 9.0 Hz, 1H), 3.80 (s, 2H), 3.66 (t, J = 12.1 Hz, 1H), 3.41 (d, J = 13.0 Hz, 1H), 2.42 (s, 3H), 1.83 (s, 3H), 1.34 (s, 3H). |
| 5 | 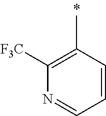 | 443.43 | 444.30 [M + H]⁺ | CD₃OD: δ 8.90 (s, 1H), 8.08 (d, J = 7.9 Hz, 1H), 7.90-7.82 (m, 2H), 7.31 (s, 1H), 6.95 (s, 1H), 4.34 (s, 1H), 4.09-4.01 (m, 1H), 4.00-3.87 (m, 1H), 3.85- 3.77 (m, 2H), 3.67 (t, J = 12.0 Hz, 1H), 3.50-3.38 (m, 1H), 1.86 (s, 3H), 1.44-1.31 (m, 3H). |
| 6 | 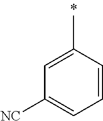 | 399.46 | 400.05 [M + H]⁺ | CDCl₃: δ 7.86-7.79 (m, 1H), 7.77 (s, 1H), 7.75-7.68 (m, 2H), 7.70-7.61 (m, 1H), 7.08 (s, 1H), 6.23 (s, 1H), 4.21-4.05 (m, 2H), 3.95-3.82 (m, 2H), 3,79 (d, J = 13.4 Hz, 1H), 3.69 (t, J = 12.0 Hz, 1H), 3.46 (t, J = 12.1 Hz, 1H), 2.08 (s, 3H), 1.39 (d, J = 6.9 Hz, 3H). |

### Example 7

### (R)-3-methyl-4-methyl-7-(1H-pyrazol-5-yl)-4-(tetrahydro-2H-pyran-4-yl)imidazo[1,5-b]pyridazin-2-yl)morpholine

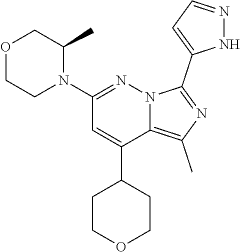 [SMILES: Cc1nc(-c2ccn[nH]2)n2nc(N3CCOC[C@H]3C)cc(C3CCOCC3)c12]

a) Preparation of 4-(3,6-dihydro-2H-pyran-4-yl)-1,2-dihydropyridazine-3,6-dione: To a solution of 4-bromo-1,2-dihydropyridazine-3,6-dione (4.0 g, 20.9 mmol), 2-(3,6-dihydro-2H-pyran-4-yl)-4,4,5,5-tetramethyl-1,3,2-dioxaborolane (8.8 g, 41.9 mmol) and 1 M aq. K₃PO₄ (62.8 mL) in DMF (40 mL) was added Pd(dppf)Cl₂ (1.5 g, 2.1 mmol) under the protection of N₂. The mixture was stirred at 100° C. for 16 h. After reaction completion, the mixture was filtered and the filtrate was evaporated to give the crude product which was washed with DCM (40 mL×3). The solid collected was dried to give the crude product (12.0 g, yellow solid) which was used for the next step directly. LC-MS(ESI): 195.05 [M+H]⁺.

b) Preparation of 4-(tetrahydro-2H-pyran-4-yl)-1,2-dihydropyridazine-3,6-dione: To a solution of 4-(3,6-dihydro-2H-pyran-4-yl)-1,2-dihydropyridazine-3,6-dione (12.0 g, 61.9 mmol, crude) in MeOH (40 mL) was added 10% Pd/C (2.0 g). The mixture was stirred at room temperature overnight under H₂ atmosphere. After reaction completion, the mixture was filtered over celite and the solid was washed with MeOH (20 mL×2). The filtrate was combined and concentrated to afford the crude product (12.0 g, gray solid) which was used for the next step directly. LC-MS(ESI): 197.05 [M+H]⁺.

c) Preparation of 3,6-dichloro-4-(tetrahydro-2H-pyran-4-yl)pyridazine: A solution of 4-(tetrahydro-2H-pyran-4-yl)-1,2-dihydropyridazine-3,6-dione (12.0 g, 61.2 mmol) in POCl₃ (100 mL) was stirred at 100° C. for 16 h, After reaction completion, the solvent was removed and the residue was quenched with water (20 mL) slowly, then a.q. ammonium hydroxide (˜25% w/w) was added to adjust pH 9. The mixture was extracted with EA (30 mL×3) and the organic phase was washed with brine, dried over anhydrous sodium sulfate and concentrated under reduced pressure to give the residue which was purified by chromatography over silica gel (petroleum ether: ethyl acetate=10:1˜1:1) to afford the target product (1.0 g, yield: 20%, 3 steps, white solid). LC-MS(EST): 233.15 [M+H]⁺.

d) Preparation of (R)-4-(6-chloro-5 -(tetrahydro-2H-pyran-4-yl)pyridazin-3-y)-3-methylmorpholine: To a solution of 3,6-dichloro-4-(tetrahydro-2H-pyran-4-yl)pyridazine (0.9 g, 3.9 mmol) in DIEA (1.5 g, 11.7 mmol) was added (R)-3-methylmorpholine (0.8 g, 7.8 mmol). The mixture was stirred at 150° C. for 6 h. After reaction completion, to the mixture was added water (10 mL) and the mixture was extracted with EA (10 mL×3). The combined organic phase was washed with brine, dried over anhydrous sodium. sulfate and concentrated under reduced pressure to afford the crude product (1.2 g, yield: 100%, black solid) which was used for the next step directly. LC-MS(ESI): 297.90 [M+H]⁺.

e) (R)-6-(3-methylmorpholino)-4-(tetrahydro-2H-pyran-4-yl)pyridazine-3-carbonitrile: To a solution of (R)-4-(6-chloro-5-(tetrahydro-2H-pyran-4-yl)pyridazin-3-yl)-3-methylmorpholine (1.3 g. 4.4 mmol) in DMF (20 mL) was added Zn(CN)₂ (1.1 g 8.8 mmol), 1,1′-bis(diphenylphosphino)ferrocene (DPPF, 0.5 g, 0.88 mmol, 0.2 eq), Pd₂(dba)₃ (0.4 g. 0.44 mmol, 0.1 eq). The mixture was stirred at 150° C. for 6 h. After reaction completion, the mixture was filtered and the filtrate was evaporated. The residue was purified by chromatography over silica gel (EA/PE, 10 to 40%) to afford the target product (1.2 g, yield: 95%, slightly yellow solid). LC-MS(ESI): 289.15 [M+H]⁺.

f) Preparation of (R)-1-(6-(3-methylmorpholino)-4-(tetrahydro-2H-pyran-4-yl)pyridazin-3-yl)ethenone: To a solution of (R)-6-(3-methylmorpholino)-4-(tetrahydro-2H-pyran-4-yl)pyridazine-3-carbonitrile (1.2 g, 4.2 mmol) in THF (12 mL) was added CH₃MgI (3.0 M in Et₂O, 12.0 mL, 36.0 mmol) under the protection of N₂ at room temperature, and the mixture was stirred for 10 min. After reaction completion, the mixture was quenched with aqueous saturated ammonium chloride (20 mL) and the mixture was extracted with EA (40 mL×3), The organic phase was separated and dried over anhydrous sodium sulfate, concentrated to give the residue which was purified by chromatography over silica gel (petroleum ether: ethyl acetate=10:1-1:1) to afford the target product (850.0 mg, yield: 67%, yellow solid). LC-MS(ESI): 306.10 [M+H]⁺.

g) Preparation of (R)-1-(6-(3-methylmorpholino)-4-(tetrahydro-2H-pyran-4-yl)pyridazin-3-yl)ethanone oxime: To a solution of (R)-1-(6-(3-methylmorpholino)-4-(tetrahydro-2H-pyran-4-yl)pyridazin-3-yl)ethanone (750.0 mg, 2.5 mmol) in MeOH (10 mL) was added hydroxylamine hydrochloride (1.8 g, 25.0 mmol) under the protection of N₂. The mixture was stirred at 70° C. overnight. After reaction completion, to the mixture was added water (10 mL) and the mixture was extracted with EA (10 mL×3). The combined organic phase was separated and dried over anhydrous sodium sulfate, concentrated to afford the crude target product (750.0 mg, yield: 9:5%, yellow solid). LC-MS(ESI): 321.35 [M+H]⁺.

h) Preparation of 1-(6-((R)-3-methylmorpholino)-4-(tetrahydro-2H-pyran-4-yl)pyridazin-3-yl)ethanamine: To a solution of (R)-1-(6-(3-methylmorpholino)-4-(tetrahydro-2H-pyran-4-yl)pyridazin-3-yl)ethanone oxime (750.0 mg, 2.4 mmol) in MeOH (10 mL) was added Raney Ni (˜15.0 g). The mixture was stirred at room temperature overnight under H₂ atmosphere. After reaction completion, the mixture was filtered over celite and the solid was washed with MeOH (10 mL×5). The filtrate was combined and concentrated to afford the crude target product (440.0 mg, gray solid). LC-MS(ESI): 307.30 [M+H]⁺.

i) Preparation of N-(1-(6-((R)-3-methylmorpholino)-4-(tetrahydro-2H-pyran-4-yl)pyridazin-3-yl)ethyl)-1H-pyrazole-5-carboxamide: To a solution of 1-(6-((R)-3-methylmorpholino)-4-(tetrahydro-2H-pyran-4-yl)pyridazin-3-yl)ethanamine (440.0 mg, 1.4 mmol) in DCM (5 mL) was added 1H-pyrazole-5-carboxylic acid (188.0 mg, 1.7 mmol), EDCI (347.6 mg, 1.8 mmol) and HOBT (19.0 mg, 0.14 mmol). The mixture was stirred at room temperature overnight. After reaction completion, to the mixture was added water (10 mL) and the mixture was extracted with DCM (10 mL×3). The combined organic phase was washed with brine, dried over anhydrous sodium sulfate and concentrated. The residue was triturated with EA (5 mL) to give the target product (380.0 mg, 2-step yield: 40%, white solid). LC-MS(ESI): 401.30 [M+H]⁺.

j) Preparation of (R)-3-methyl-4-(5-methyl-7-(1H-pyrazol-5-yl)-4-(tetrahydro-2H-pyran-4-yl)imidazo[1,5-h]pyridazin-2-yl)morpholine: A solution of N-(1-(6-((R)-3-methylmorpholino)-4-(tetrahydro-2H-pyran-4-yl)pyridazin-3-yl)ethyl)-1H-pyrazole-5-carboxamide (380.0 mg, 0.95 mmol) in POCl₃ (10 mL) was stirred at 120° C. for 4 h. After reaction completion, the solvent was removed. To the mixture was added water (10 mL) and the pH was adjusted to 8 with aq. ammonium hydroxide (25% w/w). The mixture was extracted with DCM (10 mL×2). The combined organic phase was washed with brine, dried over anhydrous sodium sulfate and concentrated. The crude product was purified by Prep-TLC (DCM:MeOH=10:1) twice to afford the target compound (40.0 mg, yield: 11%, slightly yellow solid). LC-MS(ESI): 383.35 [M+H]⁺. NMR (400 MHz, CDCl₃): δ 7.70 (s, 1H), 7.06 (s, 1H), 6.26 (s, 1H), 4.22-4.02 (m, 4H), 3.85 (s, 2H), 3.72 (t, J=13.5 Hz, 2H), 3.61 (t, J=11.9 Hz, 2H), 3.43 (t, J=12.5 Hz, 1H), 3.35-3.23 (m, 1H), 2.70 (s, 3H), 1.96-1.76 (m, 4H), 1.35 (d, J 6.8 Hz, 3H).

The following compounds of Examples 8-19 were prepared using the synthesis method similar to that described in Example 1 or Example 2.

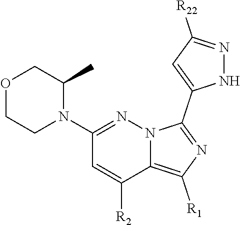

| | | | | | LC-MS | |
|---|---|---|---|---|---|---|
| Example | R₂₂ | R₁ | R₂ | MW | (ESI) | ¹H NMR (400 MHz) |
| 8 | CH₃ | CH₃ | 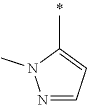 | 392.47 | 393.15 [M + H]⁺ | DMSO-d₆: δ 12.99 (s, 1H), 7.61 (s, 1H), 6.85 (s, 1H), 6.75 (s, 1H), 6.55 (s, 1H), 4.37-4.27 (m, 1H), 3.99 (d, J = 9.4 Hz, 1H), 3.88 (d, J = 12.9 Hz, 1H), 3.76-3.66 (m, 5H), 3.55 (t, J = 10.8 Hz, 1H), 3.28-3.20 (m, 1H). 2.27 (s, 3H), 1.91 (s, 3H), 1.24 (d, J = 6.5 Hz, 3H). |
| 9 | H | CH₃ | 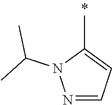 | 406.49 | 407.40 [M + H]⁺ | CDCl₃: δ 7.73 (s, 1H), 7.66 (d, J = 1.8 Hz, 1H), 7.11 (s, 1H), 6.38 (d, J = 1.8 Hz, 1H), 6.31 (s, 1H), 4.30 (p, J = 6,6 Hz, 1H), 4.18- 4.06 (m, 2H), 3.90-3.83 (m, 2H), 3.79 (d, J = 12.9 Hz, 1H), 3.69 (td, J = 11.1, 10.4, 2.3 Hz, 1H), 3.53-3.37 (m, 1H), 2.03 (s, 3H), 1.48 (s, 3H), 1.38 (d, J = 6.7 Hz, 6H). |
| 10 | H | CH₃ | 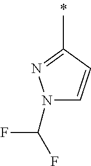 | 414.42 | 415.25 [M + H]⁺ | DMSO-d₆: δ 8.47 (d, J = 2.6 Hz, 1H), 7.99 (s, 1H), 7.95 (s, 1H), 7.22 (d, J = 2.0 Hz, 1H), 7.19-7.13 (m, 2H), 4.43 (d, J = 6.0 Hz, 1H), 4.01-3.94 (m, 2H), 3.77-3.73(m, 1H), 3.70- 3.66 (m, 1H), 3.53-3.50 (m, 1H), 3.32-3.27 (m, 1H), 2.46 (s, 3H), 1.25 (d, J = 6.7 Hz, 3H). |
| 11 | H | CH₃ | 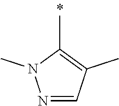 | 392.47 | 393.4 [M + H]⁺ | CDCl₃: δ 7.71 (d, J = 1.9 Hz, 1H), 7.45 (s, 1H), 7.08 (d, J = 1.9 Hz, 1H), 6.26 (d, J = 1.3 Hz, 1H), 4.11 (dd, J = 11.0, 4.1 Hz, 2H), 3.86 (s, 2H), 3.82-3.76 (m, 1H), 3.71 (s, 3H), 3.71-3.66 (m, 1H), 3.52-3.40 (m, 1H), 2.04- 1.95 (m, 6H), 1.39 (d, J = 6.8 Hz, 3H). |
| 12 | H | CH₃ | 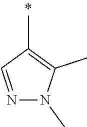 | 392.47 | 393.10 [M + H]⁺ | CDCl₃: δ 7.69 (s, 1H), 7.52 (s, 1H), 7.31 (d, J = 8.1 Hz, 1H), 7.06 (s, 1H), 6.13 (s, 1H), 4.13-4.06 (m, 2H), 3.90 (s, 3H), 3.84 (s, 2H), 3.77-3.66 (m, 2H), 3,46-3.40 (m, 1H), 2.28 (s, 3H), 2.24 (s, 3H), 1.37 (d, J = 6.7 Hz, 3H). |
| 13 | H | CH₃ | 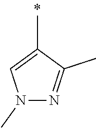 | 392.47 | 393.45 [M + H]⁺ | CDCl₃: δ 7.69 (d, J = 1.9 Hz, 1H), 7.43 (s, 1H), 7.06 (d, J = 1.9 Hz, 1H), 6.17 (s, 1H), 4.13-4.07 (m, 2H), 3.94 (s, 3H), 3.87-3.81 (m, 2H), 3.79-3.70 (m, 1H), 3.67 (dd, J = 11.7, 3.1 Hz, 1H), 3.44 (td, J = 12.4, 4.0 Hz, 1H), 2.24 (d, J = 2.8 Hz, 6H), 1.37 (d, J = 6.8 Hz, 3H). |
| 14 | H | CH₃ | 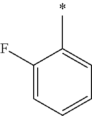 | 392.44 | 393.30 [M + H]⁺ | CDCl₃: δ 7.70 (d, J = 1.9 Hz, 1H), 7.51 (q, J = 7.2 Hz, 1H), 7.39 (t, J = 7.3 Hz, 1H), 7.31 (d, J = 7.5 Hz, 1H), 7.23 (d, J = 9.0 Hz, 1H), 7.06 (d, J = 1.9 Hz, 1H), 6.29 (s, 1H), 4.20- 4.05 (m, 2H), 3.84 (brs, 2H), 3.78 (d, J = 13.1 Hz, 1H), 3.74-3.63 (m, 1H), 3,47 (dt, J = 12.6, 6.2 Hz, 1H), 2.08 (s, 3H), 1.39 (d, |
| | | | | | | J = 6.7 Hz, 3H). |
| 15 | H | CH₃ | 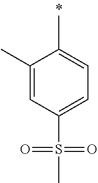 | 466.56 | 467.40 [M + H]⁺ | CD₃OD: δ 8.00 (s, 1H), 7.94 (d, J = 8.1 Hz, 1H), 7.79 (s, 1H), 7.58 (dd, J = 7.7, 2.2 Hz, 1H), 7.24 (s, 1H), 6.71 (s, 1H), 4.34 (q, J = 6.5 Hz, 1H), 4.03 (dd, J = 11.5, 3.6 Hz, 1H), 3.93 (d, J = 13.4 Hz, 1H), 3.81 (s, 2H), 3.66 (td, J = 11.8, 3.0 Hz, 1H), 3.42 (td, J = 13.4, 3.0 Hz, 1H), 3.18 (s, 3H), 2.33 (d, J = 3.8 Hz, 3H), 1.86 (s, 3H), 1.36 (t, J = 6.3 Hz, 3H). |
| 16 | H | CH₃ | 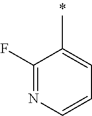 | 393.43 | 394.34 [M + H]⁺ | CDCl₃: δ 8.45-8.37 (m, 1H), 7.92-7.81 (m, 1H), 7.72 (d, J = 1.9 Hz, 1H), 7.44-7.35 (m, 1H), 7.11 (d, J = 1.9 Hz, 1H), 6.33 (s, 1H), 4.22-4.05 (m, 2H), 3.89-3.82 (m, 2H), 3.83- 3.75 (m, 1H), 3.69 (td, J = 11.7, 3.0 Hz, 1H), 3.47 (td, J = 12.5, 3.9 Hz, 1H), 2.12 (s, 3H), 1.40 (d, J = 6.7 Hz, 3H). |
| 17 | H | CH₃ | 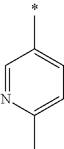 | 389.46 | 390.20 [M + H]⁺ | DMSO-d₆: δ 8.64 (d, J = 2.0 Hz, 1H), 7.97 (s, 1H), 7.92 (dd, J = 8.0, 2.3 Hz, 1H), 7.45 (d, J = 7.9 Hz, 1H), 7.22 (s, 1H), 6.96 (s, 1H), 4.41-4.39 (m, 1H), 3,98-3.97 (m, 1H), 3.96-3.95 (m, 1H), 3.74 (d, J = 11.4 Hz, 1H), 3.66 (dd, J = 11.8, 3.1 Hz, 1H), 3.52 (td, J = 12.0, 3.1 Hz, 2H), 2.56 (s, 3H), 2.08 (s, 3H), 1.24 (d, J = 6.7 Hz, 3H). |
| 18 | H | CH₃ | 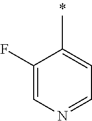 | 393.43 | 394.35 [M + H]⁺ | CDCl₃: δ 8.67 (s, 1H), 8.61 (d, J = 4.8 Hz, 1H), 7.71 (d, J = 1.9 Hz, 1H), 7.38 (t, J = 5.4 Hz, 1H), 7.08 (d, J = 1.9 Hz, 1H), 6.28 (s, 1H), 4.19-4.05 (m, 2H), 3.84 (d, J = 2.1 Hz, 2H), 3.80-3.74 (m, 1H), 3.73-3.64 (m, 1H), 3.47 (td, J = 12.7, 4.3 Hz, 1H), 2.10 (s, 3H), 1.39 (d, J = 6.8 Hz, 3H). |
| 19 | H | CH(CH₃)₂ | 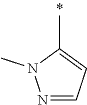 | 406.49 | 407.35 [M + H]⁺ | CDCl₃: δ 7.75-7.69 (m, 1H), 7.62 (s, 1H), 7.18-7.10 (m, 1H), 6.42 (s, 1H), 6.31 (s, 1H), 4.18-4.08 (m, 2H), 3.88-3.82 (m, 2H), 3.81- 3.73 (m, 4H), 3.72-3.62 (m, 1H), 3.46 (t, J = 11,0 Hz, 1H), 2.34-2.23 (m, 1H), 1.39 (d, J = 6.7 Hz, 3H), 1.30-1.22 (m, 3H), 1.11(s, 3H). |

### Example 20

### (R)-4-(5-fluoro-4-(1-methyl-1H-pyrazol-5-yl)-7-(1H-pyrazol-5-yl)imidazo[1,5-b]pyridazin-2-yl)-3-methylmorpholine

To a solution of (R)-3-methyl-4-(4-(1-methyl-1H-pyrazol-5-yl)-7-(1H-pyrazol-5-yl)imidazo[1,5-b]pyridazin-2-yl)morpholine (500.0 mg, 1.4 mmol) in DMF (5 mL) was added 1-fluoro-2,4,6-trimethylpyridinium tetrafluoroborate (620.0 mg, 2.8 mmol) at room temperature. The mixture was stirred at 60° C. for 5 hours. After reaction completion, the mixture was diluted with H₂O (10 mL) and extracted with DCM. (20 mL×3). The combined organic layer was washed with brine (30 mL×2), dried over Na₂SO₄, filtered and concentrated under reduced pressure to give a residue. The residue was purified by Prep-HPLC (C18, CH₃CN/H₂O, 10-40%, 0.1% HCOOH) to give the target compound (11.0 mg, yield: 2.3%, slightly yellow solid).

### Example 21

### (R)-4-(5-chloro-4-(1-methyl-1H-pyrazol-5-yl)-7-(1H-pyrazol-5-yl)imidazo[1,5-b]pyridazin-2-yl)-3-methylmorpholine

To a solution of (R)-3-methyl-4-(4-(1-methyl-1H-pyrazol-5-yl)-7-(1H-pyrazol-5-yl)imidazo[1,5-b]pyridazin-2-yl)morpholine (100.0 mg, 0.3 mmol) in DCM (5 mL) was added NCS (36.6 mg, 0.3 mmol) at −20° C. The mixture was stirred at −20° C. for 3 hours. After reaction completion, the mixture was diluted with H₂O (10 mL) and extracted with DCM (20 mL×3). The combined organic layer was washed with brine (30 mL×2), dried over Na₂SO₄, filtered and concentrated under reduced pressure to give a residue which was purified by Prep-TLC (DCM/MeOH=10:1) to give the target compound (15.0 mg, yield: 14%, yellow solid).

### Examples 22-24

a) Preparation of (R)-4-(5-bromo-4-(1-methyl-4H-pyrazol-5-yl)-7-(1H-pyrazol -5-yl)imidazo[1,5-b]pyridazin-2-yl)-3-methylmorpholine (Example 22): To a solution of (R)-3-methyl-4-(4-(1-methyl-1H-pyrazol-5-yl)-7-(1H-pyrazol-5-yl)imidazo[1,5-b]pyridazin-2-yl)morpholine (500.0 mg, 1.37 mmol) in DCM (5 mL) was added TBS (122.3 mg, 1.71 mmol) at −20° C. The mixture was stirred at −20° C. for 3 hours. After reaction completion, the mixture was diluted with H₂O (10 ML) and extracted with DCM (20 mL×3). The combined organic layers were washed with brine (30 mL×2), dried over Na₂SO₄, filtered and concentrated under reduced pressure to give a residue. The residue was purified by Prep-TLC (DCM/MeOH=20:1) to give the target product (180.0 mg, yield: 30%, yellow solid).

b) Preparation of (R)-3-methyl-4-(4-(1-methyl-1H-pyrazol-5-yl)-7-(1H-pyrazol-5-yl)-5-vinylimidazo[1,5-b]pyridazin-2-yl)morpholine (Example 23): To a solution of (R)-4-(5-bromo-4-(1-methyl-1H-pyrazol-5-yl)-7-(1H-pyrazol-5-yl)imidazo[1,5-b]pyridazin-2-yl)-3-methylmorpholine (740.0 mg, 1.7 mmol) in MAT (15 mL) was added tributyl(vinyl)stannane (1.6 g, 5.0 mmol) and Pd(PPh₃)₄ (385.3 mg, 0.3 mmol). The system was evacuated and backfilled with nitrogen three times. After stirred at 100° C. overnight, the mixture was filtered and the filtrate was concentrated under reduced pressure. The residue was purified by chromatography over silica gel (EtOAc/PE, 20 to 100%) to afford the target product (390.0 mg, yield: 60%, yellow solid).

c) Preparation of (R)-4-(5-ethyl-4-(1-methyl-4H-pyrazol-5-yl)-7-(1H-pyrazol-5-yl)imidazo[1,5-b]pyridazin-2-yl)-3-methylmorpholine (Example 24): To a solution of (R)-3-methyl-4-(4-(1-methyl-1H-pyrazol-5-yl)-7-(1H-pyrazol-5-yl)-5-vinylimidazo[1,5-b]pyridazin-2-yl)morpholine (370.0 mg, 1.0 mmol) in MeOH (20 mL) was added 10% Pd/C (160.0 mg). The system was evacuated and backfilled with H₂ three times and stirred at room temperature under H₂ balloon for 36 hours. After reaction completion, the mixture was filtered over celite and the solid was washed with MeOH (40 mL×5). The filtrate was combined and concentrated. The residue was purified by Prep-TLC (DCM:MeOH=10:1) to afford the target compound (197.5 mg, yield: 53%, yellow solid).

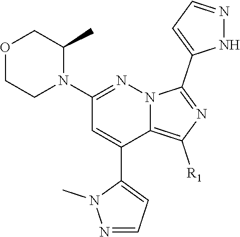

| | | | LC-MS | |
|---|---|---|---|---|
| Example | R | MW | (ESI) | ¹H NMR (400 MHz) |
| 20 | F | 382.39 | 383.05 | CD₃OD: δ 7.73 (brs, 1H), 7,61 (d, J = 2.1 Hz, 1H), 7.19 (s, 1H), 6.74 |
| | | | [M + H]⁺ | (s, 1H), 6.61 (t, J = 1.8 Hz, 1H), 4.35 (q, J = 6.3 Hz, 1H), 4.04 (dd, J = |
| | | | | 11.4, 3.7 Hz, 1H), 3.94-3.90 (m, 4H), 3.82 (d, J = 2.2 Hz, 2H), 3.66 |
| | | | | (td, J = 11.9, 3.0 Hz, 1H), 3.41 (td, J = 12.8, 3.8 Hz, 1H), 1.36 (d, J = |
| | | | | 6.7 Hz, 3H). |
| 21 | Cl | 398.85 | 399.05 | CD₃OD: δ 7.75 (d, J = 3.1 Hz, 1H), 7.60 (d, J = 1.9 Hz, 1H), 7.22- |
| | | | [M + H]⁺ | 7.17 (m, 1H), 6.83 (s, 1H), 6.53 (d, J = 1.9 Hz, 1H), 4.37~4.30 (m, |
| | | | | 1H), 4.03 (dd, J = 11.7, 3.7 Hz, 1H), 3.92 (dd, J = 13.5, 2.5 Hz, 1H), |
| | | | | 3.81 (d, J = 2.1 Hz, 2H), 3.78 (s, 3H), 3.65 (td, J = 11.8, 3.0 Hz, 1H), |
| | | | | 3.41 (td, J = 12.8, 3.8 Hz, 1H), 1.36 (d, J = 6.8 Hz, 3H) |
| 22 | Br | 443.31 | 443.30 | CD₃OD: δ 7.74 (s, 1H), 7.60 (s, 1H), 7.19 (s, 1H), 6.85 (s, 1H), 6.51 |
| | | | [M + H]⁺ | (s, 1H), 4.34 (s, 1H), 4.03 (d, J = 10.6 Hz, 1H), 3.91 (d, J = 12.8 Hz, |
| | | | | 1H), 3.80 (s, 2H), 3.76 (s, 3H), 3.65 (t, J = 11.5 Hz, 1H), 3,40 (t, J = |
| | | | | 12.3 Hz, 1H), 1.35 (d, J = 4.4 Hz, 3H). |
| 23 | 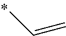 | 390.45 | 391.15 [M + H]⁺ | CD₃OD: δ 7.86-7.72 (m, 1H), 7.66 (s, 1H), 7.25 (s, 1H), 6.87-6.77 (m, 1H), 6.56 (s, 1H), 6.00 (d, J = 17.0 Hz, 1H), 5.76 (t, J = 13.7 Hz, 1H), 5.11-5.03 (m, 1H), 4.36-4.28 (m, 1H), 4.03 (d, J = 8.7 Hz, 1H), |
| | | | | 3.90 (d, J = 12.5 Hz, 1H), 3.83-3.78 (m, 2H), 3.73 (s, 3H), 3.68-3.60 |
| | | | | (m, 1H), 3.45-3.34 (m, 1H), 1.37-1.33 (m, 3H). |
| 24 | 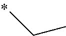 | 392.47 | 393.30 [M + H]⁺ | CDCl₃: δ 7.71 (d, J = 1.8 Hz, 1H), 7.62 (d, J = 1.8 Hz, 1H), 7.09 (s, 1H), 6.42 (d, J = 1.8 Hz, 1H), 6.29 (s, 1H), 4.17-4.05 (m, 2H), 3.84 (brs, 2H), 3.79-3.74 (m, 4H), 3.68 (m, 1H), 3.49-3.41 (m, 1H), 2.27 |
| | | | | (brs, 2H), 1.39 (d, J = 6.7 Hz, 3H), 1.06 (t, J = 7.5 Hz, 3H). |

The following compounds of Examples 25-26 were prepared using the synthesis method similar to that described in Example 23 and Example 24.

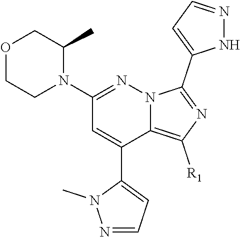

| | | | LC-MS | |
|---|---|---|---|---|
| Example | R₁ | MW | (ESI) | ¹H NMR (400 MHz) |
| 25 | 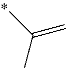 | 404.48 | 405.35 [M + H]⁺ | CDCl₃: δ 7.71 (s, 1H), 7.55 (s, 1H), 7.13 (s, 1H), 6,40 (s, 2H), 4.92 (s, 1H), 4.37 (s, 1H), 4.13 (dd, J = 24.8, 9.0 Hz, 2H), 3.85 (s, 2H), 3.79 (d, J = 13.5 Hz, 1H), 3.72-3.68 (m, 1H), 3.65 (s, 3H), 3.47 (td, J = 13.2, 4.6 Hz, 1H), 2.08 (s, 3H), 1.40 (d, J = 6.4 Hz, 3H) |
| 26 | 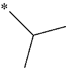 | 406.49 | 407.35 [M + H]⁺ | CDCl₃: δ 7.75-7.69 (m, 1H), 7.62 (s, 1H), 7.18-7.10 (m, 1H), 6.42 (s, 1H), 6.31 (s, 1H), 4.18-4.08 (m, 2H), 3.88- 3.82 (m, 2H), 3.81-3.73 (m, 4H), 3.72-3.62 (m, 1H), 3.46 (t, J = 11.0 Hz, 1H), 2.34-2.23 (m, 1H), 1.39 (d, J = 6.7 Hz, 3H), 1.30-1.22 (m, 3H), 1.11(s, 3H). |

### Example 27

### (R)-3-methyl-4-(4-(1-methyl-1H-pyrazol-5-yl)-7-(1H-pyrazol-3-yl)imidazo[5,1-f][1,2,4]triazin-2-yl)morpholine

The compound was prepared using the synthesis method similar to that described in Example 1, Example 2, Example 7, or using the synthesis method known to the skilled art. Yellow solid. LC-MS: 366.15 [M+1]⁺. ¹H NMR (400 MHz, CDCl₃): δ 7.94 (s, 1H), 7.74 (s, 1H), 7.65 (s, 1H), 7.15 (s, 1H), 7.09 (s, 1H), 4.70-4.57 (m, 1H), 4.28 (s, 3H), 4.18 (d, J=12.9 Hz, 1H), 4.08 (d, J=11.1 Hz, 1H), 3.90-3.75 (m, 2H), 3.65 (t, J=11.7 Hz, 1H), 3.45 (t, J=11.7 Hz, 1H), 1.41 (d, J=6.8 Hz, 3H).

### Examples 28-29

(R)-4-(5-bromo-4-(1-methyl-1H-pyrazol-5-yl)-7-(1H-pyrazol-5-yl)imidazo[5,1-f][1,2,4]triazin-2-yl)-3-methylmorpholine (Example 28) and (R)-3-methyl-4-(5-methyl-4-(1-methyl-1H-pyrazol-5-yl)-7-(1H-pyrazol-5-yl)imidazo[5,1-f][1,2,4]triazin-2-yl)morpholine (Example 29) were prepared by using the synthesis method known to the skilled art.

### Example 30

### Determination of the Inhibitory Effect of the Compounds on ATR Enzyme Activity Using ATR Enzyme Activity Experiment

ATR enzyme activity was measured using HTRF reagent (Cisbio) in a 384-well plate (Greiner, 4784075). The test compound was diluted to a 4×final concentration with reaction buffer (25 mM HEPES (pH8.0), 1.0 mM MnCl₂, 1% glycerol, 0.01% Brij-35, 5 mM DTT and 0.1% BSA), 2.5 μL of the diluted compound was added to the corresponding well, then 2.51 μL of 80 nM of p53 substrate (Eurofins, #14-952) and 2.5 ng/μL of ATR/ATRIP enzyme (Eurofins, 14-953) solution were added successively, and finally 2.5 μL of 40 μM of ATP solution was added. The mixture was centrifuged at 1000 rpm for 1 minute, and reacted at room temperature protected from light for 30 minutes. Then 5 μL of EDTA stop solution (250 mM) was added to stop the reaction. After 5 μL of detection mixture (Anti-phospho-p53 (ser15)-1((Cisbio, #61P08KAE, 0.084 ng/μL) and Anti-GST-d2 (Cisbio, #61GSTDLA, 5.00 ng/μL)) was finally added to each well, the fluorescence values at 665 nm and 615 nm were measured on the Envision 2104 instrument, Relative fluorescence ratio was calculated: Ratio_{665nm/615}-Ratio_{background}, and inhibition rate %=(1−(relative fluorescence ratio of test compound well−relative fluorescence ratio of positive control well)/(relative fluorescence ratio of blank control well−relative fluorescence ratio of positive control well))×100 was calculated. Data were analyzed using GraphPad Prism6.0, and fitted using the curve equation: Y=Bottom+(Top−Bottom)/(1+10^((LogIC₅₀-X)*HillSlope)) and IC₅₀ values were calculated.

The inhibitory effects (IC₅₀) of the compounds on ATR kinase activity were summarized in Table 1.

TABLE 1

| | | | | | | | | | | | | | | | | | | | | | | | | | | BAY- |
|---|---|---|---|---|---|---|---|---|---|---|---|---|---|---|---|---|---|---|---|---|---|---|---|---|---|---|
| Example | 1 | 2 | 3 | 4 | 5 | 6 | 7 | 8 | 9 | 10 | 11 | 12 | 13 | 14 | 15 | 16 | 17 | 18 | 20 | 21 | 22 | 23 | 24 | 25 | 26 | 1895344 |
| IC₅₀ (nM) | 1 | 3 | 2 | 3 | 3 | 5 | 1 | 4 | 3 | 217 | 2 | 1 | 14 | 10 | 6 | 9 | 18 | 10 | 41 | 6 | 2 | 2 | 2 | 1 | 1 | 42 |

Therefore, as determined by the ATR enzyme activity experiment, the disclosure compounds have good inhibitory effect on ATR kinase activity.

### Example 31

### Determination of the Inhibitory Effect of the Compounds on the Proliferation of Human Lung Cancer NCI-H460 Cell Using MTT Assay

The thawed human lung cancer NCI-H460 cells were cultured and passaged until they grew well and had a confluence about 90%, and then they were used for experiments. The cells were digested by trypsinase and centrifuged at 800 rpm for 5 minutes, the supernatant was discarded, and the residual was resuspended with fresh medium 0640 medium+10% FIBS) and counted.

The cells were seeded into 96-well cell culture plate with a density of 2000/4000 cells per well and incubated overnight in a 5% CO₂ incubator at 37° C. The stock solutions of the test substances (including the test compounds and the reference compound BAY-1895344) were serially diluted to 8 concentrations by DMSO at the ratios of 1:3 and 1:10, respectively. 5 μL diluent of each concentration was added to 120 μL of medium (25 times diluted) and mixed by shaking. The overnight cell plates were taken and the culture medium was removed, 195 μL of fresh medium was added to each well, and 5 μL of diluted medium containing the corresponding concentration of the test compound was added respectively (the final concentration of DMSO is 1%), and the culture plate was then placed in a 5% CO₂ incubator at 37° C. for 4 days. After removing the original solution, 100 μL of fresh serum-free DMEM medium containing MTT (0.5 mg/mL) was added to each well, the culture was continued. After 4 hours, the original solution was removed, 100 μL of DMSO was added to each well, the 96-well plates were shaken for 10 minutes in the dark and placed in a multi-function reader to read the absorbance at the wavelength of 552/690 nm. Cell viability (%)=(OD_{compound}−OD_{background})/(OD_{DMSO}−OD_{background})×100. GraphPad Prism 6.0 was used to analyze the data. The inhibitory activity of compounds on cell proliferation was plotted based on cell viability and the logarithm of compound concentration. The IC₅₀ value was fitted by a sigmoidal dose response curve equation Y−100/(1+10^(LogC−LogIC₅₀)), wherein C was the concentration of compound.

The inhibitory effect (IC₅₀) of the compounds on the proliferation of human lung cancer NCI-H₄₆₀ cell were summarized in Table 2.

TABLE 2

| Example | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| IC₅₀ (nM) | 16.53 | 21.91 | 27.29 | 13.03 | 17.80 | 21.79 | 15.70 | 14.89 | 30.21 | 97.32 | 30.08 | 28.00 | 33.23 | 32.00 | 12.50 |

| | | | | | | | | | | | | | BAY- |
|---|---|---|---|---|---|---|---|---|---|---|---|---|---|
| Example | 16 | 17 | 18 | 19 | 20 | 21 | 22 | 23 | 24 | 25 | 26 | 27 | 1895344 |
| IC₅₀ (nM) | 12.49 | 45.84 | 27.96 | 99.10 | 89.94 | 24.12 | 17.07 | 32.04 | 28.03 | 8.64 | 22.81 | 68.34 | 21.99 |

Therefore, as determined by MIT assay, the disclosure compounds have good inhibitory effect on the proliferation of NCI-H460 cell.

### Example 32

### Determination of the Inhibitory Effect of the Compounds on the Proliferation of Human Colon Cancer LoVo Cell Using MTT Assay

The thawed human colon cancer LoVo cells were cultured and passaged until they grew well and had a confluence about 90%, and then they were used for experiments. The cells were digested by trypsinase and centrifuged at 800 rpm for 5 minutes, the supernatant was discarded, and the residual was resuspended with fresh medium (1640 medium+10% FBS) and counted. The cells were seeded into 96-well cell culture plate with a density of 2000/4000 cells per well and incubated overnight in a 5% CO₂ incubator at 37° C. The stock solutions of the test substances (including the test compounds and the reference compound BAY-1895344) were serially diluted to 8 concentrations by DMSO at the ratios of 1:3 and 1:10, respectively. 5 μL diluent of each concentration was added to 120 μL of medium (25 times diluted) and mixed by shaking. The overnight cell plates were taken and the culture medium was removed, 195 μL of fresh medium was added to each well, and 5 μL of diluted medium containing the corresponding concentration of the test compound was added respectively (the final concentration of DMSO is 1%), and the culture plate was then placed in a 5% CO₂ incubator at 37° C. for 4 days. After removing the original solution, 100 SEL of fresh serum-free DMEM medium containing MTT (0.5 mg/mL) was added to each well, the culture was continued. After 4 hours, the original solution was removed, 100 μL of DMSO was added to each well, the 96-well plates were shaken for 10 minutes in the dark and placed in a multi-function reader to read the absorbance at the wavelength of 552/690 nm. Cell viability (%)=(OD_{compound}−OD_{background})/OD_{DMSO}−OD_{background})×100. GraphPad Prism 6.0 was used to analyze the data. The inhibitory activity of compounds on cell proliferation was plotted based on cell viability and the logarithm of compound concentration. The IC_{5.0} value was fitted by a sigmoidal dose response curve equation Y=100/(1+10^(LogC-LogIC₅₀)), wherein C was the concentration of compound.

The inhibitory effect (1050) of compounds on the proliferation of human colon cancer LoVo cell were summarized in Table 3.

TABLE 3

| Example | 1 | 2 | 3 | 4 | 5 | 6 | 7 | 8 | 9 | 10 | 18 | 12 | 13 | 14 | 15 |
|---|---|---|---|---|---|---|---|---|---|---|---|---|---|---|---|
| IC₅₀ (nM) | 26.74 | 43.57 | 49.49 | 26.89 | 25.50 | 44.10 | 24.62 | 17.87 | 39.27 | 116.10 | 41.77 | 19.65 | 46.46 | 26.24 | 19.50 |

| | | | | | | | | | | | | | BAY- |
|---|---|---|---|---|---|---|---|---|---|---|---|---|---|
| Example | 16 | 17 | 18 | 19 | 20 | 21 | 22 | 23 | 24 | 25 | 26 | 27 | 1895344 |
| IC₅₀ (nM) | 17.62 | 49.06 | 47.20 | 99.33 | 79.07 | 22.44 | 24.10 | 39.08 | 38.81 | 10.67 | 27.44 | 110.5 | 27.09 |

Therefore, as determined by MTT assay, the disclosure compounds have good inhibitory effect on the proliferation of LoVo cell.

### Example 33

### Pharmacokinetic Studies of the Compounds Following Single Oral Administration to Mice

The compound of the present invention was formulated into a 0.5% methylcellulose/water uniform suspension, and was administered to CD-1 (ICR) mice by gavage at 10 mg/kg. Plasma samples were collected at 8 time points of 0.250, 0.500, 1.00, 2.00, 4.00, 6.00, 8.00 and 24.0 hours post-dose. Concentrations of the compound were determined by LC-MS/MS method.

The mouse pharmacokinetic parameters of the compounds were summarized in Table 4.

TABLE 4

| | | Cₘₐₓ | AUC₀₋ₜ | AUC_{0-inf} |
|---|---|---|---|---|
| Example | t_{1/2} (h) | (ng · mL⁻¹) | (ng · h · mL⁻¹) | (ng · h · mL⁻¹) |
| 1 | 1.64 | 9343 | 98507 | 98517 |
| 4 | 3.2 | 9008 | 43376 | 43951 |
| 5 | 3.42 | 8933 | 30826 | 31662 |
| 14 | 3.75 | 10787 | 77348 | 78719 |
| 21 | 2.65 | 7343 | 42772 | 42925 |
| 22 | 5.88 | 7673 | 59010 | 63023 |
| 23 | 1.66 | 6608 | 22356 | 24116 |
| 24 | 5.40 | 15367 | 161501 | 171092 |
| 25 | 2.56 | 16867 | 70013 | 70195 |
| 26 | 5.82 | 7180 | 46439 | 48979 |
| a | 4.46 | 3023 | 12427 | 17986 |
| b | 0.5 | 713 | 2604 | 2779 |
| c | 3.71 | 2377 | 23051 | 26374 |
| d | 5.82 | 3130 | 14885 | 24053 |

Note:
1) t_{1/2}: Elimination half-life; Cₘₐₓ: Maximum plasma concentration; AUC₀₋ₜ: Area under the plasma concentration-time curve from time 0 to the time of last measurable concentration; AUC_{0-inf}: Area under the plasma concentration-time curve from time 0 to infinity.
2) Examples a, b, c, and d here are Examples 13, 25, 42 and 47 in WO2020259601A1, respectively.

The results show that the compounds of the present invention have good oral absorption and high exposure in mice.

### Example 34

### Determination of the Inhibitory Effect of the Compound of Example 1 on the Proliferation of Various Human Cancer Cells Using MTT Assay

The thawed human cancer cells (including human non-small cell lung cancer A549 cell, human breast cancer HCC₁₈₀₆ cell, human colorectal adenocarcinoma cell, human ovarian cancer OVCAR-3 cell and human large cell lung cancer NCI-H460 cell) were cultured and passaged until they grew well and had a confluence about 90%, and then they were used for experiments. The cells were digested by trypsinase and centrifuged at 800 rpm for 5 minutes, the supernatant was discarded, and the residual was resuspended with fresh medium (1640 medium+10% FRS) and counted. The cells were seeded into 96well cell culture plate with a density of 2000/4000 cells per well and incubated overnight in a 5% CO₂ incubator at 37° C. The stock solutions of the test substances were serially diluted to 8 concentrations by DMSO at the ratios of 1:3 and 1:10, respectively. 5 μL diluent of each concentration was added to 120 of medium (25 times diluted) and mixed by shaking. The overnight cell plates were taken and the culture medium was removed, 195 μL of fresh medium was added to each well, and 5 μL of diluted medium containing the corresponding concentration of the test compound was added respectively (the final concentration of DMSO is 1%), and the culture plate was then placed in a 5% CO₂ incubator at 37° C for 4 days. After removing the original solution, 100 mL of fresh serum-free DMEM medium containing MTT (0.5 mg/mL) was added to each well, the culture was continued. After 4 hours, the original solution was removed, 100 μL, of DMSO was added to each well, the 96-well plates were shaken for 10 minutes in the dark and placed in a multi-function reader to read the absorbance at the wavelength of 552/690 nm. Cell viability (%)=(OD_{compound}−OD_{background})/(OD_{DMSO}−OD_{background})×100. GraphPad Prism 6.0 was used to analyze the data. The inhibitory activity of compounds on cell proliferation was plotted based on cell viability and the logarithm of compound concentration. The IC₅₀ value was fitted by a sigmoidal dose response curve equation Y=100/(1+10^(LogC−LogIC₅₀)), wherein C was the concentration of compound.

The inhibitory effect data (IC₅₀) of the compound of Example 1 on the proliferation of human cancer cells were summarized in Table 5.

TABLE 5

| | A549 | HCC1806 | HCT116 | OVCAR-3 | NCI-H460 |
|---|---|---|---|---|---|
| IC₅₀/nM | 156.70 | 38.81 | 22.48 | 181.60 | 19.02 |

Therefore, as determined by MIT assay, the compound of Example 1 have good inhibitory effect on the proliferation of various human cancer cells.

### Example 35

### Determination of the Synergistic Inhibition of the Compound of Example 1 in Combination With Senaparib on the Proliferation of Human Ovarian Cancer OVCAR-3 Cell Using MTT Assay

The thawed human ovarian cancer OVCAR-3 cells were cultured and passaged until they grew well and had a confluence about 90%, and then they were used for experiments. The cells were digested by trypsinase and centrifuged at 800 rpm for 5 minutes, the supernatant was discarded, and the residual was resuspended with fresh medium (1640 medium+10% FBS) and counted. The cells were seeded into 96-well cell culture plate with an appropriate density and incubated overnight in a 5% CO₂ incubator at 37° C. The stock solutions of the test substances were serially diluted to 4-5 concentrations by DMSO at the ratios of 1:3, respectively. 5 UL diluent of each concentration was added to 120 μL of medium (25 times diluted) and mixed by shaking. The overnight cell plates were taken and the culture medium was removed, 190 μL of fresh medium was added to each well, and 5 μL of diluted compound or 5 μL of medium containing DMSO was added respectively (the final concentration of DMSO is 1%), and the culture plate was then placed in a 5% CO₂ incubator at 37° C. for 3 days. After removing the original solution, 100 μL of fresh serum-free DMEM medium containing MTT (0.5 mg/mL) was added to each well, the culture was continued. After 4 hours, the original solution was removed, 100 μL of DMSO was added to each well, the 96-well plates were shaken for 10 minutes in the dark and placed in a multi-function reader to read the absorbance at the wavelength of 552/690 nm. Cell viability (%)=(OD_{compound}−OD_{background})/(OD_{DMSO}−OD_{background})×100. The combination index (CI) was calculated with the software CalcuSyn.

The CI values of the compound of Example 1 in combination with senaparib were summarized in Table 6.

TABLE 6
Combination Index (CI)
CI Value

| | 11 nM | 33 nM | 100 nM | 300 nM |
|---|---|---|---|---|
| | Example 1 | Example 1 | Example 1 | Example 1 |
| 3000 nM senaparib | 0.044 | 0.12 | 0.364 | 1.195 |
| 1000 nM senaparib | 0.052 | 0.148 | 0.424 | 1.276 |
| 333 nM senaparib | 0.069 | 0.18 | 0.488 | 1.377 |
| 111 nM senaparib | 0.215 | 0.213 | 0.544 | 1.471 |
| 37 nM senaparib | 1.157 | 0.275 | 0.603 | 1.588 |

Note:
CI < 0.1 means a strong synergistic effect; 0.1 < CI < 1 means a synergistic effect, and CI > 1 means no synergistic effect.

The experimental results show that the combination of the compound of Example 1 and senaparib has good synergistic effect on inhibiting the proliferation of human ovarian cancer OVCAR-3 cells.

Having now fully described this disclosure, it will be understood by those of ordinary skill in the art that the same can be performed within a wide and equivalent range of conditions, formulations and other parameters without affecting the scope of the disclosure or any embodiment thereof. All patents, patent applications and publications cited herein are fully incorporated by reference herein in their entirety.

1. A compound of Formula I: 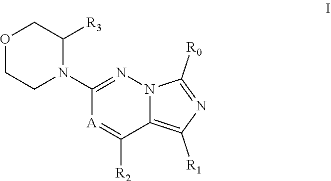
or stereoisomers, tautomers, N-oxides, hydrates, solvates, isotope-substituted derivatives, or pharmaceutically acceptable salts thereof, or mixtures thereof, or prodrugs thereof, wherein:
A is N or CH;
R₀ is an optionally substituted aryl, an optionally substituted heterocyclic group, an optionally substituted carbocyclic group, an optionally substituted heteroaryl, an optionally heteroaryl alkyl, 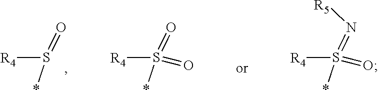
wherein * indicates an attachment position of the group to the rest of the compound;
R₁ is halogen, an optionally substituted C₁-C₆ alkyl, an optionally substituted C₃-C₆ cycloalkyl, an optionally substituted C₂-C₆ alkenyl, or an optionally substituted C₂-C₆ alkynyl;
R₂ is halogen, C₁-C₆ alkyl, C₁-C₆ alkoxy, C₂-C₆ alkenyl, C₂-C₆ alkynyl, carbocyclic group, heterocyclic group, aryl, heteroaryl, —(SO)R₄, —(SO₂)R₄, —SR₄, —NR₆R₇, —(CO)R₆, —(CO)OR₆, —(CO)NR₆R₇, —(SO₂)NR₆R₇, —NR₆(SO₂)R₄, —((SO)—NR₅)R₈, —N═(SO)R₄R₈, —SiR₅R₈R₉, —(PO)(OR₆)₂, —(PO)(OR₆)R₈ or —(PO)(R₈)₂, wherein the said C₁-C₆ alkyl, C₁-C₆ alkoxy, C₃-C₆ cycloalkyl, C₂-C₆ alkenyl, C₂-C₆ alkynyl, carbocyclic group, heterocyclic group, aryl and heteroaryl each are optionally substituted;
R₃ is hydrogen or an optionally substituted C₁-C₆ alkyl;
R₄ is an optionally substituted alkyl or an optionally substituted alkylaryl;
R₅ is hydrogen, an optionally substituted alkyl, —(CO)OR₆ or —(CO)NR₆R₇;
R₆ and R₇ are independently hydrogen, an optionally substituted C₁-C₁₀ alkyl, an optionally substituted carbocyclic group, an optionally substituted heterocyclic group, an optionally substituted aryl or an optionally substituted heteroaryl; or R₆ and R₇ together with the N and C to which they are attached form an optionally substituted 4-7 membered cyclic amino group, which optionally comprises one or more additional heteroatoms selected from O, N and S;
R₈ is C₁-C₄ alkyl, or in the case of —N═(SO)R₄R₅, R₄ and R₅ together with the S to which they attached form a 5-8 membered heterocycloalkyl; and
R₉ is hydrogen or C₁-C₄ alkyl.

2. The compound of claim 1, wherein the compound is a compound of Formula II: 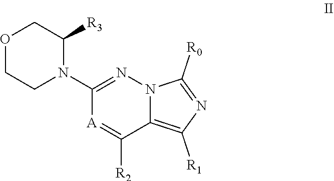
or stereoisomers, tautomers, N-oxides, hydrates, solvates, isotope-substituted derivatives, or pharmaceutically acceptable salts thereof, or mixtures thereof, or prodrugs thereof.

3. The compound of claim 1, or stereoisomers, tautomers, N-oxides, hydrates, isotope-substituted derivatives, solvates or pharmaceutically acceptable salts thereof, or mixtures thereof, or prodrugs thereof, wherein:
A is CH;
R₀ is an optionally substituted alkylsulfonyl, an optionally substituted aryl, an optionally substituted heterocyclic group, an optionally substituted carbocyclic group or an optionally substituted heteroaryl;
R₁ is halogen, C₁-C₆ alkyl, C₃-C₆ cycloalkyl or C₂-C₆ alkenyl optionally substituted with 1-6 substituents selected from halogen, hydroxyl and —NRₐR_{b}, wherein the said Rₐ and R_{b} are independently hydrogen or C₁-C₄ alkyl;
R₂ is an optionally substituted carbocyclic group, an optionally substituted heterocyclic group, an optionally substituted aryl, or an optionally substituted heteroaryl; and
R₃ is C₁-C₄ alkyl.

4. The compound of claim 2, or stereoisomers, tautomers, N-oxides, hydrates, isotope-substituted derivatives, solvates or pharmaceutically acceptable salts thereof, or mixtures thereof, or prodrugs thereof, wherein:
A is CH;
R₀ is sulfonyl substituted with C₁-C₄ alkyl; or pyrazolyl, pyrrolyl, or imidazolyl optionally substituted with 1 or 2 substituents selected from a group consisting of C₁-C₄ alkyl, halogen, hydroxyl, C₁-C₄ alkoxy and amino;
R₁ is halogen, C₁-C₄ alkyl, C₃-C₄ cycloalkyl or C₂-C₄ alkenyl;
R₂ is phenyl optionally substituted with 1 or 2 substituents selected from C₁-C₆ alkyl, halogen, cyano and sulfonyl substituted with C₁-C₄ alkyl; pyrazolyl or pyridyl optionally substituted with 1 or 2 substituents selected from C₁-C₆ alkyl, haloC₁-C₆ alkyl and halogen; or tetrahydropyranyl optionally substituted with 1 or 2 substituents selected from C₁-C₆ alkyl, haloC₁-C₆ alkyl and halogen; and
R₃ is C₁-C₃ alkyl.

5. The compound of claim 1, wherein the compound is a compound of Formula III: 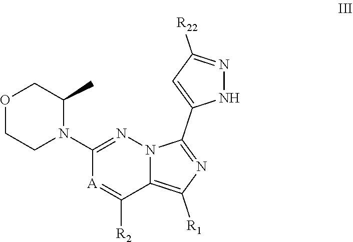
or stereoisomers, tautomers, N-oxides, hydrates, solvates, isotope-substituted derivatives, or pharmaceutically acceptable salts thereof, or mixtures thereof, or prodrugs thereof, wherein:
R₂₂ is hydrogen, halo or an optionally substituted C₁-C₆ alkyl.

6. The compound of claim 5, wherein:
A is CH;
R₂₂ is hydrogen or C₁-C₃ alkyl;
R₁ is halogen, C₁-C₄ alkyl, C₃-C₆ cycloalkyl or C₂-C₄ alkenyl; and
R₂ is carbocyclic group, heterocyclic group, aryl, or heteroaryl, which are optionally substituted by 1-3 substituents selected from the group consisting of C₁-C₆ alkyl, halo C₁-C₆ alkyl, cyano, halogen and sulfonyl substituted with C₁-C₄ alkyl.

7. The compound of claim 1, or stereoisomers, tautomers, N-oxides, hydrates, isotope-substituted derivatives, solvates or pharmaceutically acceptable salts thereof, or mixtures thereof, or prodrugs thereof, wherein R₂ is phenyl optionally substituted with 1 or 2 substituents selected from C₁-C₆ alkyl, halogen, cyano and sulfonyl substituted with C₁-C₄ alkyl; pyrazolyl or pyridyl optionally substituted with 1 or 2 substituents selected from C₁-C₆ alkyl, haloC₁-C₆ alkyl and halogen; or tetrahydropyranyl optionally substituted with 1 or 2 substituents selected from C₁-C₆ alkyl, haloC₁-C₆ alkyl and halogen.

8. The compound of claim 1, wherein the compound is a compound of Formula IV: 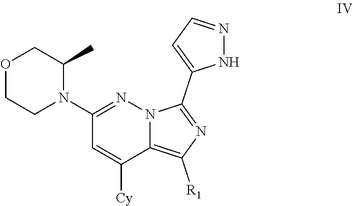
or stereoisomers, tautomers, N-oxides, hydrates, solvates, isotope-substituted derivatives, or pharmaceutically acceptable salts thereof, or mixtures thereof, or prodrugs thereof, wherein:
Cy is carbocyclic group, heterocyclic group, aryl, heteroaryl, —NR₆R₇, —NR₆(SO₂)R₄, or —N═(SO)R₄R₈, wherein, the said carbocyclic group, heterocyclic group, aryl and heteroaryl can be each optionally substituted;
R₄ is an optionally substituted alkyl or an optionally substituted alkylaryl;
R₆ and R₇ are each independently hydrogen, an optionally substituted C₁-C₁₀ alkyl, an optionally substituted cycloalkyl, an optionally substituted aryl or an optionally substituted heteroaryl; or R₆ and R₇ together to which they are attached form an optionally substituted 4-7 membered cyclic amino group, which optionally comprises one or more additional heteroatoms selected from O, N and S; and
R₈ is C₁-C₄ alkyl, or in the case of —N═(SO)R₃R₇, R₃ and R₇ together to which they attached form a 5-8 membered heterocycloalkyl.

9. The compound of claim 8, or stereoisomers, tautomers, N-oxides, hydrates, isotope-substituted derivatives, solvates or pharmaceutically acceptable salts thereof, or mixtures thereof, or prodrugs thereof, wherein:
R₁ is halogen, C₁-C₄ alkyl, C₃-C₆ cycloalkyl or C₂-C₄ alkenyl; and
Cy is phenyl optionally substituted with 1 or 2 substituents selected from C₁-C₆ alkyl, halogen, cyano and sulfonyl substituted with C₁-C₄ alkyl; pyrazolyl or pyridyl optionally substituted with 1 or 2 substituents selected from C₁-C₆ alkyl, haloC₁-C₆ alkyl and halogen; or tetrahydropyranyl optionally substituted with 1 or 2 substituents selected from C₁-C₆ alkyl, haloC₁-C₆ alkyl and halogen.

10. The compound of claim 8, or stereoisomers, tautomers, N-oxides, hydrates, isotope-substituted derivatives, solvates or pharmaceutically acceptable salts thereof, or mixtures thereof, or prodrugs thereof, wherein A is CH or N; R₁ is halogen, C₁-C₃ alkyl, C₃-C₄ cycloalkyl or C₂-C₃ alkenyl; Cy is phenyl optionally substituted with 1 or 2 substituents selected from C₁-2 alkyl, C₁-2 alkylsulfonyl and cyano, pyridyl optionally substituted with 1 or 2 substituents selected from halogen, C₁-2 alkyl, and halo C₁-2 alkyl, or pyrazolyl optionally substituted with C₁-2 alkyl substituted at one N atom of the pyrazolyl.

11. The compound of claim 1, wherein the compound is selected from the group consisting of:
(R)-3-methyl-4-(5-methyl-4-(1-methyl-1H-pyrazol-5-yl)-7-(1H-pyrazol-5-yl)imidazo[1,5-b]pyridazin-2-yl)morpholine;
(R)-4-(4-(1-ethyl-1H-pyrazol-5-yl)-5-methyl-7-(1H-pyrazol-5-yl)imidazo[1,5-b]pyridazin-2-yl)-3-methylmorpholine;
(R)-3-methyl-4-(5-methyl-7-(1H-pyrazol-5-yl)-4-(o-tolyl)imidazo[1,5-b]pyridazin-2-yl)morpholine;
(R)-3-methyl-4-(5-methyl-4-(2-methylpyridin-3-yl)-7-(1H-pyrazol-5-yl)imidazo[1,5-b]pyridazin-2-yl)morpholine;
(R)-3-methyl-4-(5-methyl-7-(1H-pyrazol-5-yl)-4-(2-(trifluoromethyl)pyridin-3-yl)imidazo[1,5-b]pyridazin-2-yl)morpholine;
(R)-3-(5-methyl-2-(3-methylmorpholino)-7-(1H-pyrazol-5-yl)imidazo[1,5-b]pyridazin-4-yl)benzonitrile;
(R)-3-methyl-4-(5-methyl-7-(1H-pyrazol-5-yl)-4-(tetrahydro-2H-pyran-4-yl)imidazo[1,5-b]pyridazin-2-yl)morpholine;
(R)-3 -methyl-4-(5-methyl-4-(1-methyl-1H-pyrazol-5-yl)-7-(3 -methyl-1H-pyrazol-5-yl)imidazo[1,5-b]pyridazin-2-yl)morpholine;
(R)-4-(4-(1-isopropyl-1H-pyrazol-5-yl)-5-methyl-7-(1H-pyrazol-5-yl)imidazo[1,5-b]pyridazin-2-yl)-3-methylmorpholine;
(R)-4-(4-(1-(difluoromethyl)-1H-pyrazol-3-yl)-5-methyl-7-(1H-pyrazol-5-yl)imidazo[1,5-b]pyridazin-2-yl)-3-methylmorpholine;
(R)-4-(4-(1,4-dimethyl-1H-pyrazol-5-yl)-5-methyl-7-(1H-pyrazol-5-yl)imidazo[1,5-b]pyridazin-2-yl)-3-methylmorpholine;
(R)-4-(4-(1,5-dimethyl-1H-pyrazol-4-yl)-5-methyl-7-(1H-pyrazol-5-yl)imidazo[1,5-b]pyridazin-2-yl)-3-methylmorpholine;
(R)-4-(4-(1,3-dimethyl-1H-pyrazol-4-yl)-5-methyl-7-(1H-pyrazol-5-yl)imidazo[1,5-b]pyridazin-2-yl)-3-methylmorpholine;
(R)-4-(4-(2-fluorophenyl)-5-methyl-7-(1H-pyrazol-5-yl)imidazo[1,5-b]pyridazin-2-yl)-3-methylmorpholine;
(R)-3-methyl-4-(5-methyl-4-(2-methyl-4-(methylsulfonyl)phenyl)-7-(1H-pyrazol-5-yl)imidazo[1,5-b]pyridazin-2-yl)morpholine;
(R)-4-(4-(2-fluoropyridin-3-yl)-5-methyl-7-(1H-pyrazol-5-yl)imidazo[1,5-b]pyridazin-2-yl)-3-methylmorpholine;
(R)-3-methyl-4-(5-methyl-4-(6-methylpyridin-3-yl)-7-(1H-pyrazol-5-yl)imidazo[1,5-b]pyridazin-2-yl)morpholine;
(R)-4-(4-(3-fluoropyridin-4-yl)-5-methyl-7-(1H-pyrazol-5-yl)imidazo[1,5-b]pyridazin-2-yl)-3-methylmorpholine;
(R)-4-(4-(1-(difluoromethyl)-1H-pyrazol-5-yl)-5-methyl-7-(1H-pyrazol-5-yl)imidazo[1,5-b]pyridazin-2-yl)-3-methylmorpholine;
(R)-4-(5-fluoro-4-(1-methyl-1H-pyrazol-5-yl)-7-(1H-pyrazol-5-yl)imidazo[1,5-b]pyridazin-2-yl)-3-methylmorpholine;
(R)-4-(5-chloro-4-(1-methyl-1H-pyrazol-5-yl)-7-(1H-pyrazol-5-yl)imidazo[1,5-b]pyridazin-2-yl)-3-methylmorpholine;
(R)-4-(5-bromo-4-(1-methyl-1H-pyrazol-5-yl)-7-(1H-pyrazol-5-yl)imidazo[1,5-b]pyridazin-2-yl)-3-methylmorpholine;
(R)-3-methyl-4-(4-(1-methyl-1H-pyrazol-5-yl)-7-(1H-pyrazol-5-yl)-5-vinylimidazo[1,5-b]pyridazin-2-yl)morpholine;
(R)-4-(5-ethyl-4-(1-methyl-1H-pyrazol-5-yl)-7-(1H-pyrazol-5-yl)imidazo[1,5-b]pyridazin-2-yl)-3-methylmorpholine;
(R)-3-methyl-4-(4-(1-methyl-1H-pyrazol-5-yl)-5-(prop-1-en-2-yl)-7-(1H-pyrazol-5-yl)imidazo[1,5-b]pyridazin-2-yl)morpholine;
(R)-4-(5-isopropyl-4-(1-methyl-1H-pyrazol-5-yl)-7-(1H-pyrazol-5-yl)imidazo[1,5-b]pyridazin-2-yl)-3-methylmorpholine;
(R)-4-(5-bromo-4-(1-methyl-1H-pyrazol-5-yl)-7-(1H-pyrazol-5-yl)imidazo[5,1-f][1,2,4]triazin-2-yl)-3-methylmorpholine; and
(R)-3-methyl-4-(5-methyl-4-(1-methyl-1H-pyrazol-5-yl)-7-(1H-pyrazol-5-yl)imidazo[5,1-f][1,2,4]triazin-2-yl)morpholine;
or a stereoisomer, a tautomer, a N-oxide, a hydrate, an isotope-substituted derivative, a solvate or a pharmaceutically acceptable salt thereof, or a mixture thereof.

12.-15. (canceled)

16. A pharmaceutical composition comprising the compound of claim 1 and a pharmaceutically acceptable carrier.

17. The compound of claim 3, or stereoisomers, tautomers, N-oxides, hydrates, isotope-substituted derivatives, solvates or pharmaceutically acceptable salts thereof, or mixtures thereof, or prodrugs thereof, wherein:
R₀ is sulfonyl substituted with C₁-C₄ alkyl; or pyrazolyl, pyrrolyl, or imidazolyl optionally substituted with 1 or 2 substituents selected from a group consisting of C₁-C₄ alkyl, halogen, hydroxyl, C₁-C₄ alkoxy and amino; and
R₂ is phenyl optionally substituted with 1 or 2 substituents selected from C₁-C₆ alkyl, halogen, cyano and sulfonyl substituted with C₁-C₄ alkyl; pyrazolyl or pyridyl optionally substituted with 1 or 2 substituents selected from C₁-C₆ alkyl, haloC₁-C₆ alkyl and halogen; or tetrahydropyranyl optionally substituted with 1 or 2 substituents selected from C₁-C₆ alkyl, haloC₁-C₆ alkyl and halogen.

18. The compound of claim 7, or stereoisomers, tautomers, N-oxides, hydrates, isotope-substituted derivatives, solvates or pharmaceutically acceptable salts thereof, or mixtures thereof, or prodrugs thereof, wherein R₂ is:

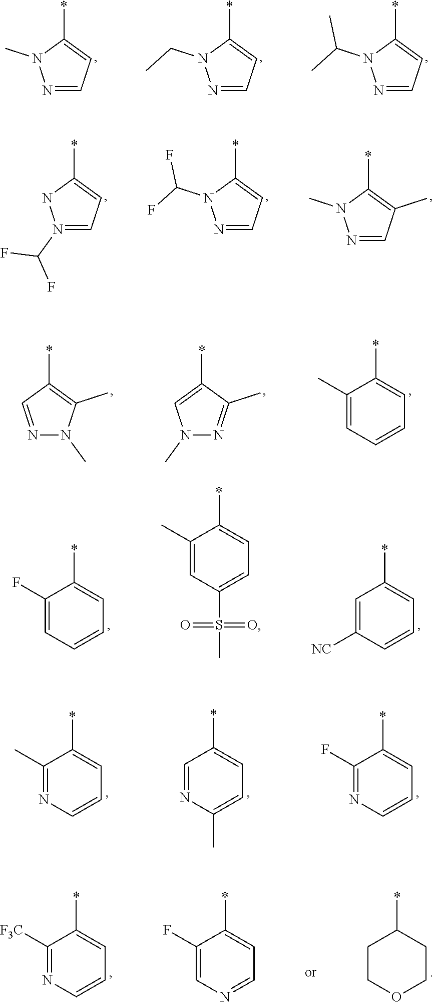

19. A method for treatment or prevention of an ATR kinase mediated disease, comprising administering to a subject the compound of claim 1 or a stereoisomer, a tautomer, a N-oxide, a hydrate, an isotope-substituted derivative, a solvate or a pharmaceutically acceptable salt thereof, or a mixture thereof or a prodrug thereof.

20. The method according to claim 19, wherein the disease is cancer.

21. The method according to claim 20, wherein the cancer is selected from the group consisting of liver cancer, melanoma, Hodgkin's disease, non-Hodgkin's lymphomas, acute lymphocytic leukemia, chronic lymphocytic leukemia, multiple myeloma, neuroblastoma, breast carcinoma, ovarian carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, non small-cell lung carcinoma, small-cell lung carcinoma, stomach carcinoma, colon carcinoma, malignant pancreatic insulinoma, malignant carcinoid carcinoma, choriocarcinoma, mycosis fungoide, head and neck carcinoma, osteogenic sarcoma, pancreatic carcinoma, acute granulocytic leukemia, hairy cell leukemia, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

22. The method according to claim 20, further comprising administering to the subject at least one known anticancer drug or a pharmaceutically acceptable salt thereof selected from the group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximab, Blinatumomab, ipilimumab, avastin, herceptin, mabthera, imatinib, gefitinib, erlotinib, osimertinib, afatinib, ceritinib, alectinib, crizotinib, erlotinib, lapatinib, sorafenib, sunitinib, nilotinib, dasatinib, pazopanib, torisel, everolimus, vorinostat, romidepsin, panobinostat, belinostat, tamoxifen, letrozole, fulvestrant, mitoguazone, octreotide, retinoic acid, arsenic trioxide, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine), palbociclib, olaparib, niraparib, rucaparib, talazoparib, pamiparib, fluzoparib and senaparib.

23. The method according to claim 19, wherein the method is used in combination with radiotherapy.

24. The pharmaceutical composition according to claim 16, wherein the composition further comprises at least one known anticancer drug or pharmaceutically acceptable salts thereof selected from the group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximab, Blinatumomab, ipilimumab, avastin, herceptin, mabthera, imatinib, gefitinib, erlotinib, osimertinib, afatinib, ceritinib, alectinib, crizotinib, erlotinib, lapatinib, sorafenib, sunitinib, nilotinib, dasatinib, pazopanib, torisel, everolimus, vorinostat, romidepsin, panobinostat, belinostat, tamoxifen, letrozole, fulvestrant, mitoguazone, octreotide, retinoic acid, arsenic trioxide, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine), palbociclib, olaparib, niraparib, rucaparib, talazoparib, pamiparib, fluzoparib and senaparib.
